SEQUENCE LISTING (19 sequences)

SEQ ID NO: 1 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 3 - protein, 7 aa, Simian virus 40
PKKKRKV

SEQ ID NO: 4 - protein, 10 aa, Simian virus 40
PKKKRKVEAS

SEQ ID NO: 5 - protein, 16 aa, Artificial Sequence
  Synthetic
KRPAATKKAGQAKKKK

SEQ ID NO: 6 - protein, 9 aa, Homo sapiens
PAAKRVKLD

SEQ ID NO: 7 - protein, 11 aa, Homo sapiens
RQRRNELKRSP

SEQ ID NO: 8 - protein, 38 aa, Homo sapiens
NQSSNFGPMKGGNFGGRSSGPYGGGGQYFAKPRNQGGY

SEQ ID NO: 9 - protein, 42 aa, Homo sapiens
RMRIZFKNKGKDTAELRRRRVEVSVELRKAKKDEQILKRRNV

SEQ ID NO: 10 - protein, 8 aa, Homo sapiens
VSRKRPRP

SEQ ID NO: 11 - protein, 8 aa, Homo sapiens
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis D virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

(19) United States
(12) Patent Application Publication
(10) Pub. No.: US 2025/0327128 A1
(43) Pub. Date: Oct. 23, 2025

(54) PARTIAL-EMT SIGNATURE FOR PREDICTION OF HIGH-RISK HISTOPATHOLOGIC FEATURES AND CANCER OUTCOMES ACROSS DEMOGRAPHIC POPULATIONS

(71) Applicants: Washington University, St. Louis, MO (US); The Broad Institute, Inc., Cambridge, MA (US); Massachusetts Institute of Technology, Cambridge, MA (US)

(72) Inventors: Sidharth Puram, St. Louis, MO (US); Angela Mazul, St. Louis, MO (US); Aviv Regev, Cambridge, MA (US)

(21) Appl. No.: 18/271,191

(22) PCT Filed: Jan. 6, 2022
(86) PCT No.: PCT/US2022/011397

§ 371 Date: Jul. 6, 2023

Related U.S. Application Data
(60) Provisional application No. 63/134,491, filed on Jan. 6, 2021.

(51) Int. Cl. C12Q 1/6886; G01N 33/574

(52) U.S. Cl. CPC C12Q 1/6886; G01N 33/57407; C12Q 2600/118; C12Q 2600/136; C12Q 2600/158; G01N 2500/04

(57) ABSTRACT

The present invention advantageously provides for use of a p-EMT signature for the treatment and prognosis of head and neck cancer across demographic groups. The p-EMT signature is differentially expressed across demographic groups. The p-EMT state indicates a high risk of metastasis and adverse clinical features that may be used to direct treatment of head and neck cancer.

### CROSS-REFERENCE TO RELATED APPLICATIONS

This application claims the benefit of U.S. Provisional Application No. 63/134,491 filed Jan. 6, 2021. The entire contents of the above-identified application are hereby fully incorporated herein by reference.

### REFERENCE TO AN ELECTRONIC SEQUENCE LISTING

The contents of the electronic sequence listing (“BROD-5300WP_ST25.txt”; Size is 7,928 bytes and it was created on Jan. 4, 2022) is herein incorporated by reference in its entirety.

### TECHNICAL FIELD

The subject matter disclosed herein is generally directed to methods of using the expression of a p-EMT signature to stratify and treat subjects suffering from head and neck squamous cell carcinoma (HNSCC) and belonging to specific demographic groups.

### BACKGROUND

Head and neck squamous cell carcinoma (HNSCC) is associated with significant morbidity and mortality, the majority of which is associated with heavy tobacco and alcohol use. The incidence of HPV-associated oropharyngeal cancer is rapidly increasing, and the survival of non-HPV-associated HNSCC is plateauing. Although HNSCC incidence has been decreasing due to tobacco cessation efforts, the burden of head and neck cancer remains high in neighborhoods with low socioeconomic status and among racial and ethnic minorities, suggesting that the decline in HNSCC may not be uniform across locales and sociodemographic groups. Additionally, HNSCC survival has not improved dramatically, especially among low socioeconomic and minority groups. The majority of deaths in HNSCC are related to metastasis and treatment failure after traditional multi-modal therapy, with salvage therapies including immune checkpoint inhibitors exhibiting poor overall response rates. Non-Hispanic Black patients are more likely to fail treatment than non-Hispanic White patients, highlighting the importance of access to care, treatment adherence, and external support to head and neck cancer survival. The ability to treat HNSCC is primarily limited by an incomplete understanding of the molecular pathways that drive metastasis and treatment failure (Puram S V, Rocco J W. Molecular Aspects of Head and Neck Cancer Therapy. Hematol Oncol Clin North Am. 2015; 29(6):971-92), and how these pathways potentially underlie racial health disparities. Due to the head and neck region's complexity, oncologic outcomes must be carefully balanced against exuberant primary or adjuvant treatment, which may compromise quality of life.

Given the morbidity and mortality associated with advanced HNSCC and persistent health disparities, there is an urgent need to more effectively stratify patients based on molecular markers and to develop novel therapeutics that more effectively and equitably combat these tumors, with implications for other cancers in which metastasis and treatment resistance remains a challenge. Unfortunately, defining high-risk and low-risk with a biomarker in HNSCC populations apriori remains difficult.

HNSCC has a high degree of genetic and epigenetic intra-tumoral heterogeneity compared to other tumors (Puram, et al., 2015), primarily reflecting chronic alcohol and tobacco exposure in most patients. The high degree of intra-tumoral heterogeneity in HNSCC is a significant impediment to overcoming treatment resistance. This intra-tumoral heterogeneity is an essential predictor of HNSCC patient outcomes, but the mechanisms by which this heterogeneity contributes to disease progression have remained largely unknown (Gotte K, et al., Intratumoral genomic heterogeneity in advanced head and neck cancer detected by comparative genomic hybridization. Adv Otorhinolaryngol. 2005; 62:38-48; Hass H G, et al., DNA ploidy, proliferative capacity and intratumoral heterogeneity in primary and recurrent head and neck squamous cell carcinomas (HNSCC)—potential implications for clinical management and treatment decisions. Oral Oncol. 2008; 44(1):78-85; Zhang X C, et al., Tumor evolution and intratumor heterogeneity of an oropharyngeal squamous cell carcinoma revealed by whole-genome sequencing. Neoplasia. 2013; 15(12):1371-8; Mroz E A, Rocco J W. MATH, a novel measure of intratumor genetic heterogeneity, is high in poor-outcome classes of head and neck squamous cell carcinoma. Oral Oncol. 2013; 49(3):211-5; Mroz E A, et al., High intratumor genetic heterogeneity is related to worse outcome in patients with head and neck squamous cell carcinoma. Cancer. 2013; 119(16):3034-42; and Mroz E A, Rocco J W. Intra-tumor heterogeneity in head and neck cancer and its clinical implications. World journal of otorhinolaryngology—head and neck surgery. 2016; 2(2):60-7). A range of bulk sequencing analyses have attempted to characterize HNSCC broadly, but the considerable intra-tumoral heterogeneity in HNSCC represents a challenge to existing efforts. However, emerging technologies such as single cell RNA-sequencing (scRNA-seq) have enabled the analysis of heterogeneous samples in exquisite detail, allowing for the comprehensive identification of discrete populations of malignant, stromal, and immune cells, including rare cell populations which may drive clinically relevant phenotypes. The first single-cell RNA-seq analysis of HNSCC has identified a partial-EMT (p-EMT) program at the leading edge of tumors which triggers invasion and can be a potential predictor of nodal metastasis and adverse histopathologic features (Puram S V, Tirosh I, Parikh A S, et al. Single-Cell Transcriptomic Analysis of Primary and Metastatic Tumor Ecosystems in Head and Neck Cancer. Cell. 2017; 171(7):1611-1624.e24; and Parikh A S, Puram S V, Faquin W C, et al., Immunohistochemical quantification of partial-EMT in oral cavity squamous cell carcinoma primary tumors is associated with nodal metastasis. Oral Oncol. 2019; 99:104458).

Although the biological relationship between p-EMT programs and aggressive tumors has been well established (Parikh, et al., 2019; Wangmo C, et al., Epithelial-Mesenchymal Transition Predicts Survival in Oral Squamous Cell Carcinoma. Pathol Oncol Res. 2020; 26(3):1511-8; and Kisoda S, et al., Prognostic value of partial EMT-related genes in head and neck squamous cell carcinoma by a bioinformatic analysis. Oral Dis. 2020), no studies have characterized the p-EMT program's clinical relevance with epidemiologic rigor within populations underrepresented in research. Molecular prognostication may have different outcomes within race/ethnic populations, signifying the importance of considering race/ethnicity when developing a biomarker. Given the potential of p-EMT as a prognostic biomarker, there is a need to determine if p-EMT programs are associated with poor clinical features and outcomes and if p-EMT interacts with race. There is also a need to determine whether p-EMT can be used to stratify patients of different demographic groups to provide for effective therapies.

Citation or identification of any document in this application is not an admission that such a document is available as prior art to the present invention.

### SUMMARY

In one aspect, the present invention provides for a method of treating an epithelial cancer comprising determining whether a subject suffering from an epithelial cancer belongs to a high or low risk group by: detecting an average expression of one or more partial EMT-like (p-EMT) signature genes or polypeptides in malignant cells from the subject, wherein the one or more p-EMT signature genes or polypeptides are selected from the group consisting of SERPINE1, TGFB1, MMP10, LAMC2, P4HA2, PDPN, ITGA5, LAMA3, CH13, TNC, MMP2, EMP3, INHBA, LAMB3, SNAIL2, and VIM; and comparing the average expression of the subject p-EMT signature genes or polypeptides to a control average expression of the p-EMT signature genes or polypeptides for malignant cells obtained from a plurality of subjects having the epithelial cancer and belonging to the same demographic group as the subject, wherein the subject is in a high risk group if the average expression in the subject is higher than the control average expression for the demographic group, and the subject is in the low risk group if the average expression in the subject is lower than the control average expression for the demographic group; and if the subject is in a low risk group, then treating the subject with a treatment that comprises immunotherapy, neoadjuvant therapy and/or chemoradiation; if the subject is in a high risk group, then treating the subject with a treatment that comprises lymph node dissection, adjuvant chemotherapy, adjuvant radiation or post-operative radiation treatment (PORT), chemoradiation, neoadjuvant and/or adjuvant immunotherapy, administering an agent that inhibits TGF beta signaling; and/or administering one or more agents targeting malignant cells expressing a p-EMT signature, optionally, further comprising treating the subject with immunotherapy, neoadjuvant therapy and/or chemoradiation. In certain embodiments, one p-EMT signature gene is detected. In certain embodiments, two p-EMT signature genes are detected. In certain embodiments, three p-EMT signature genes are detected. In certain embodiments, four p-EMT signature genes are detected. In certain embodiments, five p-EMT signature genes are detected. In certain embodiments, six p-EMT signature genes are detected. In certain embodiments, seven p-EMT signature genes are detected. In certain embodiments, eight p-EMT signature genes are detected. In certain embodiments, nine p-EMT signature genes are detected. In certain embodiments, ten p-EMT signature genes are detected. In certain embodiments, eleven p-EMT signature genes are detected. In certain embodiments, twelve p-EMT signature genes are detected. In certain embodiments, thirteen p-EMT signature genes are detected. In certain embodiments, fourteen p-EMT signature genes are detected. In certain embodiments, fifteen p-EMT signature genes are detected. In certain embodiments, all sixteen p-EMT signature genes are detected. In certain embodiments, the demographic group is selected from the group consisting of African American, Caucasian, non-Caucasian, non-smoker, current smoker, former smoker, male and female. In certain embodiments, the control average expression is the median average expression of the one or more p-EMT signature genes or polypeptides for malignant cells obtained from the plurality of tumors for the demographic group; or wherein the control average expression level is an intermediate average expression level of the one or more p-EMT signature genes or polypeptides within the range of average expression for malignant cells obtained from the plurality of tumors for the demographic group.

In certain embodiments, the average expression is determined by RNA sequencing (RNA-seq). In certain embodiments, the average expression is determined by RNA-seq of bulk tumor cells and inference of malignant cell expression. In certain embodiments, the average expression is determined by single cell RNA-seq. In certain embodiments, the average expression is determined by detecting the one or more polypeptides using immunohistochemistry (IHC). In certain embodiments, the one or more polypeptides detected by IHC are selected from the group consisting of PDPN, LAMC2, LAMB3, MMP10, TGFBI and ITGA5. In certain embodiments, detecting the average expression further comprises determining the percentage of cells having an average expression higher than the control average expression for the demographic group, wherein the subject is in the high risk group if the percentage of cells having a higher average expression is greater than a control percentage and the subject is in the low risk group if the percentage of cells having a higher average expression is lower than a control percentage. For example, the high risk group can have greater than 1, 5, 10, 20, 30, 40 or 50% of cells having a higher average expression and the low risk group can have less than 1, 5, 10, 20, 30, 40 or 50% of cells having a higher average expression (e.g., 0%).

In certain embodiments, the method further comprises determining a p-EMT score for the subject, wherein the p-EMT score is the difference between the average expression of the one or more p-EMT signature genes or polypeptides (e.g., 1, 2, 3, 4, 5, 6, 7, 8, 9, 10, 11, 12, 13, 14, 15, or 16 genes or polypeptides) and the average expression of a control gene set for the subject, wherein the control gene set comprises genes having a similar distribution of expression levels as the control average expression for each p-EMT signature gene or polypeptide, wherein a p-EMT high score is greater than zero (e.g., 0.1, 0.2, 0.3, 0.4, 0.5, 0.6, 0.7, 0.8, 0.9, 1.0, 2.0 or more) and a p-EMT low score is less than zero (e.g., −0.1, −0.2, −0.3, −0.4, −0.5, −0.6, −0.7, −0.8, −0.9, −1.0, −2.0 or less), and wherein the subject is in the high risk group if a p-EMT high score is detected and the subject is in the low risk group if a p-EMT low score is detected. In certain embodiments, the control gene set has at least 20-100 genes for each p-EMT gene, such as 20, 30, 40, 50, 60, 70, 80, 90, 100, 200, 300 or more control genes. In certain embodiments, a p-EMT high score is greater than 0.5 (e.g., 0.5-1.0, 0.5-2.0) and a p-EMT low score is less than −0.5 (e.g., −0.5-−1.0, −0.5-−2.0) for any demographic selected from the group consisting of Caucasian, non-smoker and female. In certain embodiments, a p-EMT high score is greater than 0.4 (e.g., 0.4-0.9, 0.4-1.9) and a p-EMT low score is less than −0.4 (e.g., −0.4-−0.9, −0.4-−1.9) for non-Caucasians. In certain embodiments, a p-EMT high score is greater than 0.3 (e.g., 0.3-0.8, 0.3-1.8) and a p-EMT low score is less than −0.3 (e.g., −0.3-−0.8, −0.3-−1.8) for males. In certain embodiments, a p-EMT high score is greater than 0.2 (e.g., 0.2-0.7, 0.2-1.7) and a p-EMT low score is less than −0.2 (e.g., −0.2-−0.7, −0.2-−1.7) for African Americans. In certain embodiments, a p-EMT high score is greater than 0.1 (e.g., 0.1-0.6, 0.1-1.6) and a p-EMT low score is less than −0.1 (e.g., −0.1-−0.6, −0.1-−1.6) for African American males.

In certain embodiments, the subject has a clinically N0 (cN0) neck. In certain embodiments, the p-EMT signature is detected at diagnosis. In certain embodiments, the subject is older than 35, 40, 45, 50, 55 or 60 years old. In certain embodiments, the subject was diagnosed for human papilloma virus (HPV).

In another aspect, the present invention provides for a method of stratifying subjects suffering from an epithelial cancer and belonging to a demographic group into high and low risk groups comprising detecting an average expression of one or more partial EMT-like (p-EMT) signature genes or polypeptides in malignant cells from a subject in need thereof, said signature comprising one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM; and comparing the average expression of the subject p-EMT signature genes or polypeptides to a control average expression of the p-EMT signature genes or polypeptides for malignant cells obtained from a plurality of subjects having the epithelial cancer and belonging to the same demographic group as the subject, wherein the subject is in the high risk group if the average expression in the subject is higher than the control average expression for the demographic group, and the subject is in the low risk group if the average expression in the subject is lower than the control average expression for the demographic group. In certain embodiments, the demographic group is selected from the group consisting of African American, Caucasian, non-Caucasian, non-smoker, current smoker, former smoker, male and female. In certain embodiments, the control average expression is the median average expression of the one or more p-EMT signature genes or polypeptides for malignant cells obtained from the plurality of tumors for the demographic group; or wherein the control average expression level is an intermediate average expression level of the one or more p-EMT signature genes or polypeptides within the range of average expression for malignant cells obtained from the plurality of tumors for the demographic group.

In certain embodiments, the average expression is determined by RNA sequencing (RNA-seq). In certain embodiments, the average expression is determined by RNA-seq of bulk tumor cells and inference of malignant cell expression. In certain embodiments, the average expression is determined by single cell RNA-seq. In certain embodiments, the average expression is determined by detecting the one or more polypeptides using immunohistochemistry (IHC). In certain embodiments, the one or more polypeptides detected by IHC are selected from the group consisting of PDPN, LAMC2, LAMB3, MMP10, TGFBI and ITGA5. In certain embodiments, detecting the average expression further comprises determining the percentage of cells having an average expression higher than the control average expression for the demographic group, wherein the subject is in the high risk group if the percentage of cells having a higher average expression is greater than a control percentage and the subject is in the low risk group if the percentage of cells having a higher average expression is lower than a control percentage.

In certain embodiments, the method further comprises determining a p-EMT score for the subject, wherein the p-EMT score is the difference between the average expression of the one or more p-EMT signature genes or polypeptides and the average expression of a control gene set for the subject, wherein the control gene set comprises genes having a similar distribution of expression levels as the control average expression for each p-EMT signature gene or polypeptide, wherein a p-EMT high score is greater than zero and a p-EMT low score is less than zero, and wherein the subject is in the high risk group if a p-EMT high score is detected and the subject is in the low risk group if a p-EMT low score is detected. In certain embodiments, the control gene set has at least 20-100 genes for each p-EMT gene. In certain embodiments, a p-EMT high score is greater than 0.5 and a p-EMT low score is less than −0.5 for any demographic selected from the group consisting of Caucasian, non-smoker and female. In certain embodiments, a p-EMT high score is greater than 0.4 and a p-EMT low score is less than −0.4 for non-Caucasians. In certain embodiments, a p-EMT high score is greater than 0.3 and a p-EMT low score is less than −0.3 for males. In certain embodiments, a p-EMT high score is greater than 0.2 and a p-EMT low score is less than −0.2 for African Americans. In certain embodiments, a p-EMT high score is greater than 0.1 and a p-EMT low score is less than −0.1 for African American males.

In certain embodiments, the subject has a clinically N0 (cN0) neck. In certain embodiments, the p-EMT signature is detected at diagnosis. In certain embodiments, the subject is older than 35, 40, 45, 50, 55 or 60 years old. In certain embodiments, the subject was diagnosed for human papilloma virus (HPV).

In certain embodiments, the high risk group has decreased survival as compared to the low risk group. In certain embodiments, the high risk group is at least twice as likely to die in a 15 year period as compared to all other subjects. In certain embodiments, the high risk group has increased risk for occult nodal metastasis as compared to the low risk group. In certain embodiments, the high risk group has increased risk for perineural invasion (PNI) as compared to the low risk group.

In certain embodiments, chemoradiation comprises cisplatin. In certain embodiments, the immunotherapy comprises checkpoint blockade therapy.

In another aspect, the present invention provides for a method of monitoring a subject undergoing treatment for an epithelial cancer comprising determining whether the p-EMT signature or p-EMT score according to any embodiment herein increases or decreases in the subject during the treatment. In certain embodiments, the treatment is an agent that inhibits TGF beta signaling.

In another aspect, the present invention provides for a method for identifying an agent capable of modulating or shifting a p-EMT signature comprising applying a candidate agent to a cell or population of cells having a p-EMT signature comprising one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM; and detecting modulation of the p-EMT signature for the cell or cell population by the candidate agent, wherein the p-EMT signature is detected according to any embodiment herein.

In certain embodiments, the epithelial cancer is selected from the group consisting of head and neck cancer (HNSCC), lung, breast, prostate, colon, cutaneous squamous cell carcinoma and esophageal carcinoma. In certain embodiments, the epithelial cancer is head and neck cancer (HNSCC).

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-FIG. 1B—p-EMT predicts survival better than smoking. FIG. 1A. Results with a sociodemographically diverse cohort. (left) Graph showing survival probability of subjects that are p-EMT high and p-EMT low. (right) Table showing the survival hazard ratio for the indicated patient factors. FIG. 1B. Results with OCSCC TCGA tumors. Kaplan-Meier survival curve by p-EMT expression among malignant cells. (Inset) Adjusted hazard ratios (HR).

FIG. 2—p-EMT predicts poor clinical features. Graphs showing the percentage of indicated clinical features in p-EMT low and p-EMT high tumors (T stage—T2 and T4, N stage—N0 and N+, margin −/+, PNI −/+, LVI −/+, and grade—grade 1/2 and grade 3).

FIG. 3—p-EMT predicts poor clinical features. (top) Table showing odds ratio N+ using the indicated prognostic factor (p-EMT high, PNI, High grade). (bottom) Graph showing the fraction of surviving patients in p-EMT low and p-EMT high tumors.

FIG. 4A-FIG. 4B—p-EMT predicts occult metastasis. FIG. 4A. Schematic depicting neck dissection. FIG. 4B. (top) Graph showing justified neck dissections (i.e., a tumor was found) in relation to p-EMT score. (bottom) Table showing percent of node negative and node positive tumors in p-EMT low and p-EMT high tumors.

FIG. 5—Gender and race interact to influence survival in head and neck cancer. Kaplan-Meier survival curve by race and gender. (Inset) Restricted mean survival time (RMST).

FIG. 6—Schematic of experimental approach.

FIG. 7A-FIG. 7B—Overall survival by pEMT signature based on previously determined cutpoints for FIG. 7A. larynx and FIG. 7B. oral cavity cancer.

FIG. 8A-FIG. 8D—Survival by race. FIG. 8A. Disease free survival by tertile categories of p-EMT for Caucasian subjects at risk as determined by pEMT expression. FIG. 8B. Overall survival by tertile categories of p-EMT for Caucasian subjects at risk as determined by pEMT expression. FIG. 8C. Disease free survival by tertile categories of p-EMT for African American subjects at risk as determined by pEMT expression. FIG. 8D. Overall survival by tertile categories of p-EMT for African American subjects at risk as determined by pEMT expression.

FIG. 9A-FIG. 9D—Survival by gender. FIG. 9A. Disease free survival by tertile categories of p-EMT for male subjects at risk as determined by pEMT expression. FIG. 9B. Overall survival by tertile categories of p-EMT for male subjects at risk as determined by pEMT expression. FIG. 9C. Disease free survival by tertile categories of p-EMT for female subjects at risk as determined by pEMT expression. FIG. 9D. Overall survival by tertile categories of p-EMT for female subjects at risk as determined by pEMT expression.

FIG. 10A-FIG. 10F—Survival by smoking status. FIG. 10A. Disease free survival by tertile categories of p-EMT for current smokers at risk as determined by pEMT expression. FIG. 10B. Overall survival by tertile categories of p-EMT for current smokers at risk as determined by pEMT expression. FIG. 10C. Disease free survival by tertile categories of p-EMT for former smokers at risk as determined by pEMT expression. FIG. 10D. Overall survival by tertile categories of p-EMT for former smokers at risk as determined by pEMT expression. FIG. 10E. Disease free survival by tertile categories of p-EMT for non-smokers at risk as determined by pEMT expression. FIG. 10F. Overall survival by tertile categories of p-EMT for non-smokers at risk as determined by pEMT expression.

FIG. 11A-FIG. 11C—Overall survival Kaplan-Meier curves in high p-EMT black subjects, high p-EMT white subjects, low p-EMT black subjects, and low p-EMT white subjects. FIG. 11A. all sites, FIG. 11B. larynx cancer and FIG. 11C. oral cavity cancer.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. patent application Ser. No. 16/604,651, filed Apr. 12, 2018 and published as US20200071773A1; and International Patent Application PCT/US2018/027383, filed Apr. 12, 2018 and published as WO2018191553A1. Reference is also made to Puram S V, Tirosh I, Parikh A S, et al. Single-Cell Transcriptomic Analysis of Primary and Metastatic Tumor Ecosystems in Head and Neck Cancer. Cell. 2017; 171(7):1611-1624.e24. doi:10.1016/j.cell.2017.10.044; Puram S V, Parikh A S, Tirosh I. Single cell RNA-seq highlights a role for a partial EMT in head and neck cancer. Mol Cell Oncol. 2018; 5(3):e1448244. Published 2018 Mar. 7. doi:10.1080/23723556.2018.1448244; and Parikh A S, Puram S V, Faquin W C, et al. Immunohistochemical quantification of partial-EMT in oral cavity squamous cell carcinoma primary tumors is associated with nodal metastasis. Oral Oncol. 2019; 99:104458. All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide for use of a p-EMT signature to stratify head and neck cancer patients into high risk and low risk groups based on demographic groups. Moreover, embodiments disclosed herein provide for treating the patients based on their risk group. As used herein, head and neck cancer can be used interchangeably with head and neck squamous cell carcinoma (HNSCC) or oral cavity squamous cell carcinoma (OCSCC). Oral cavity squamous cell carcinoma (OCSCC) mortality is rising rapidly, especially among low socioeconomic populations, compared to nearly all other cancers. Due to the head and neck region's complexity, oncologic outcomes must be carefully balanced against exuberant primary or adjuvant treatment, which may compromise quality of life (e.g., neck dissection). Beyond these biologic and functional challenges, OCSCC demonstrates substantial cancer health disparities by socioeconomic status.

Unfortunately, defining high-risk and low-risk patients with a biomarker in OCSCC populations a priori remains difficult. Despite advances in treatment, survival improvements in OCSCC have stagnated with no molecular prognosticators and a high degree of health disparities. Current molecular markers are mainly being developed in homogeneous, high socioeconomic status populations. Therefore, it is critical to develop a prognosticator in a diverse population and account for relevant health equities early on in biomarker development to reduce health disparities. Currently, there are no biomarkers that risk-stratify HNSCC outside of histopathology. Additionally, most biomarkers are developed in homogenous cohorts with limited validation in diverse populations, such as those of St. Louis. Applicants address this urgent need by developing a predictive biomarker to guide clinical decision-making and account for potential health disparities in OCSCC. Specifically, Applicants provide for use of a p-EMT biomarker across multiple populations to identify high and low risk patients who may be candidates for treatment intensification or de-intensification, respectively, while challenging existing treatment paradigms by integrating tumor genomics to more accurately predict outcomes and treatment needs.

OCSCC tumors are intrinsically heterogeneous compared to other cancers, with chronic tobacco and alcohol exposure further amplifying intra-tumoral heterogeneity in many patients. To comprehensively define intra-tumoral heterogeneity, Applicants completed the first single cell RNA-sequencing (scRNA-seq) analysis of OCSCC (Puram et al., 2017). Among the diverse malignant programs, Applicants identified a partial epithelial-to-mesenchymal transition (p-EMT) program. This program is distinct from traditional EMT; p-EMT cells express some mesenchymal markers (e.g. Vimentin) and EMT transcription factors (Snail2), yet retain epithelial marker expression. This p-EMT program localizes at the leading edge of tumors where it appears to trigger invasion.

Applicants provide analyses herein that demonstrate that p-EMT is associated with overall survival, disease-free survival, and nodal metastasis while considering cancer health disparities at the outset to maximize the impact of cancer genomic research and health equity. Importantly, p-EMT is differential by race and a stronger predictor of death among Black Americans (African American) than White Americans (Caucasian American). Additionally, p-EMT is more prognostic than smoking, stage, age, or tumor subsite, suggesting a robust underlying biologic effect of p-EMT signaling. Thus, p-EMT can reliably predict unfavorable biology in diverse HNSCC patients better than existing histopathologic criteria and be differential by race and socioeconomic status and thus a mediator in OCSCC health disparities. In other words, the present invention provides for treating specific demographic groups, such as African Americans, by detecting a p-EMT signature in the specific demographic group and treating based on the high p-EMT or low p-EMT expression. In addition, the present invention provides for treating specific HNSCC cancers, such as laryngeal cancer or oral cavity cancer, by detecting a p-EMT signature in a subject having a cancer in the specific location and treating based on the high p-EMT or low p-EMT expression. In addition, the present invention provides for treating HPV-negative oropharyngeal cancer by detecting a p-EMT signature in a subject having a HPV-negative oropharyngeal cancer and treating based on the high p-EMT or low p-EMT expression.

p-EMT can be predicted based on bulk RNA-seq data followed by deconvolution. Additionally, detecting several p-EMT marker genes by IHC can potentially match the performance of next-generation sequencing approaches in a socioeconomically diverse population and within racial subgroups. Applicants can also examine the relationship between p-EMT and sociodemographic factors and as a mediator for health disparities.

Applicants have established a prospectively collected, clinically annotated, diverse cohort of OCSCC tumors. Applicants can perform bulk RNA-seq on the 400 tumors in the diverse cohort, of which 200 are from Black American patients, in which the RNA-seq data can be deconvolved using previously developed computational algorithms to determine a malignant p-EMT score. Applicants hypothesized that p-EMT is related to high-risk histopathologic features and cancer outcomes and that p-EMT can improve the prediction of occult nodal metastasis and the need for neck dissection in cN0 patients. Finally, Applicants can create a tissue microarray used for immunohistochemistry (IHC) of the top ten p-EMT markers and determine which markers correlate with the genomic-based p-EMT score within overall and all racial subgroups to extend the generalizability of the biomarker.

Given the diversity of St. Louis and the available cohort, Applicants can investigate if p-EMT expression is different based on gender, race, and socioeconomic status. Applicants can calculate the contribution of sociodemographic factors to p-EMT score with a principal component score for significant factors. Next, Applicants can use regression modeling to estimate the effect of spatial and individual-level variables on p-EMT signature. Finally, Applicants can conduct survival analyses to determine how the p-EMT marker interacts with sociodemographics to influence survival. The p-EMT scoring can then be adjusted for distinct sociodemographic groups.

In certain embodiments, the methods described herein may be used for any epithelial cancer. Studies have suggested that EMT is a process that occurs in all epithelial tumors. In certain embodiments, epithelial tumors all express similar p-EMT programs as described herein. HNSCC is one of many common epithelial tumors. In certain embodiments, detection of the p-EMT signature described herein in any epithelial tumor predicts 1) risk of having lymph node or distant metastasis, 2) tumor stage, 3) adverse pathologic features, 4) need for adjuvant (radiation/chemotherapy) treatment, 5) treatment response, and 6) overall survival. The examples described herein show that the p-EMT signature is a strong genetic predictor of having lymph node (LN) involvement and that the signature predicts the need for a neck dissection (removal of LN).

Cancers may include, but are not limited to, breast cancer, colon cancer, lung cancer, prostate cancer, testicular cancer, brain cancer, skin cancer, rectal cancer, gastric cancer, esophageal cancer, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, cutaneous squamous cell carcinoma, carcinomas, sarcomas, and soft tissue cancers. Thus, the disclosure is generally applicable to any type of cancer in which expression of an EMT program occurs. In certain embodiments, the signature is useful for all epithelial tumors, including but not limited to lung, breast, prostate, colon, cutaneous squamous cell carcinoma and esophageal carcinoma.

### Stratifying Head and Neck Cancer Subjects by Risk

p-EMT Signature

In certain embodiments, the detection of a partial EMT (p-EMT) signature in malignant cells from a subject suffering from a head and neck cancer can predict high-risk histopathologic features and cancer outcomes. As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, p-EMT can be referred to as a biomarker. In certain embodiments, the p-EMT biomarker refers to the average expression of the p-EMT genes in the signature (described further herein). In certain embodiments, the p-EMT biomarker refers to a metagene. As used herein a “metagene” refers to a pattern or aggregate of gene expression and not an actual gene. Each metagene may represent a collection or aggregate of genes behaving in a functionally correlated fashion within the genome. The p-EMT biomarker may also refer to an average intensity of staining in IHC. Applicants identified that the p-EMT signature is a better predictor of survival risk than all other pathological features currently used. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

In certain embodiments, the p-EMT signature is a better predictor in specific demographic groups. In certain embodiments, the p-EMT score is more predictive in African American subjects or subjects identifying as having African heritage. In certain embodiments, the p-EMT score is more predictive in male subjects. In certain embodiments, the p-EMT score is more predictive in African American male subjects or male subjects identifying as having African heritage. In certain embodiments, the p-EMT score is more predictive in smokers.

In certain embodiments, the p-EMT signature includes one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM; or one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, VIM, SEMA3C, PRKCDBP, ANXA5, DHRS7, ITGB1, ACTN1, CXCR7, ITGB6, IGFBP7, THBS1, PTHLH, TNFRSF6B, PDLIM7, CAV1, DKK3, COL17A1, LTBP1, COL5A2, COL1A1, FHL2, TIMP3, PLAU, LGALS1, PSMD2, CD63, HERPUD1, TPM1, SLC39A14, CIS, MMP1, EXT2, COL4A2, PRSS23, SLC7A8, SLC31A2, ARPC1B, APP, MFAP2, MPZL1, DFNA5, MT2A, MAGED2, ITGA6, FSTL1, TNFRSF12A, IL32, COPB2, PTK7, OCIAD2, TAX1BP3, SEC13, SERPINH1, TPM4, MYH9, ANXA8L1, PLOD2, GALNT2, LEPREL1, MAGED1, SLC38A5, FSTL3, CD99, F3, PSAP, NMRK1, FKBP9, DSG2, ECM1, HTRA1, SERINC1, CALU, TPST1, PLOD3, IGFBP3, FRMD6, CXCL14, SERPINE2, RABAC1, TMED9, NAGK, BMP1, ESYT1, STON2, TAGLN and GJA1. The signature does not include most classical EMT transcription factors, such as, ZEB1/2, TWIST1/2, or SNAIL1.

The signature was identified as one of 6 meta-signatures in head and neck cancer samples (Table 1). In certain embodiments, the p-EMT signature may be detected alone or in combination with any of the other signatures. In certain embodiments, a p-EMT high score is determined by detection of both a p-EMT and epithelial signature. In certain embodiments, the epithelial signature includes one or more genes or polypeptides selected from the group consisting of IL1RN, SLPI, CLDN4, CLDN7, S100A9, SPRR1B, PVRL4, RHCG, SDCBP2, S100A8, APOBEC3A, LY6D, KRT16, KRT6B, KRT6A, LYPD3, KRT6C, KLK10, KLK11, TYMP, FABP5, SCO2, FGFBP1 and JUP; or one or more genes or polypeptides selected from the group consisting of SPRR1B, KRT16, KRT6B, KRT6C, KRT6A, KLK10, KLK11 and CLDN7; or one or more genes or polypeptides selected from the group consisting of IL1RN, SLPI, CLDN4, S100A9, SPRR1B, PVRL4, RHCG, SDCBP2, S100A8, APOBEC3A, GRHL1, SULT2B1, ELF3, KRT16, PRSS8, MXD1, S100A7, KRT6B, LYPD3, TACSTD2, CDKN1A, KLK11, GPRC5A, KLK10, TMBIM1, PLAUR, CLDN7, DUOXA1, PDZK1IP1, NCCRP1, IDS, PPL, ZNF750, EMP1, CLDN1, CRB3, CYB5R1, DSC2, S100P, GRHL3, SPINTI, SDR16C5, SPRR1A, WBP2, GRB7, KLK7, TMEM79, SBSN, PIM1, CLIC3, MALATI, TRIP10, CAST, TMPRSS4, TOM1, A2ML1, MBOAT2, LGALS3, ERO1L, EHF, LCN2, YPEL5, ALDH3B2, DMKN, PIK3IP1, CEACAM6, OVOL1, TMPRSS11E, CD55, KLK6, SPRR2D, NDRG2, CD24, HIST1H1C, LY6D, CLIP1, HIST1H2AC, BNIPL, QSOX1, ECM1, DHRS3, PPP1R15A, TRIM16, AQP3, IRF6, CSTA, RAB25, HOPX, GIPC1, RAB11FIP1, CSTB, KRT6C, PKP1, JUP, MAFF, DSG3, AKTIP, KLF3, HSPB8 and H1F0; or one or more genes or polypeptides selected from the group consisting of LY6D, KRT16, KRT6B, LYPD3, KRT6C, TYMP, FABP5, SCO2, FGFBP1, JUP, PIP4, DSC2, TMBIM1, KRT14, C1QBP, SFN, S100A14, RAB38, GJB5, MRPL14, TRIM29, ANXA8L2, KRT6A, PDHB, AKR1B10, LAD1, DSG3, MRPL21, NDUFS7, PSMD6, AHCY, GBP2, TXN2, PSMD13, NOP16, EIF4EBP1, MRPL12, HSD17B10, LGALS7B, THBD, EXOSC4, APRT, ANXA8L1, ATP5G1, S100A2, TBRG4, MAL2, NHP2L1, DDX39A, ZNF750, UBE2L6, WDR74, PPIF, PRMT5, VSNL1, VPS25, SNRNP40, ADRM1, NDUFS8, TUBA1C, TMEM79, UQCRFS1, EIF3K, NME2, PKP3, SERPINB1, RPL26L1, EIF6, DSP, PHLDA2, S100A16, LGALS7, MT1X, UQCRC2, EIF3I, MRPL24, CCT7, RHOV, ECE2, SSBP1, POLDIP2, FIS1, CKMT1A, GJB3, NME1, MRPS12, GPS1, ALG3, MRPL20, EMC6, SRD5A1, PA2G4, ECSIT, MRPL23, NAA20, HMOX2, COA4, DCXR, PSMD8 and WBSCR22.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

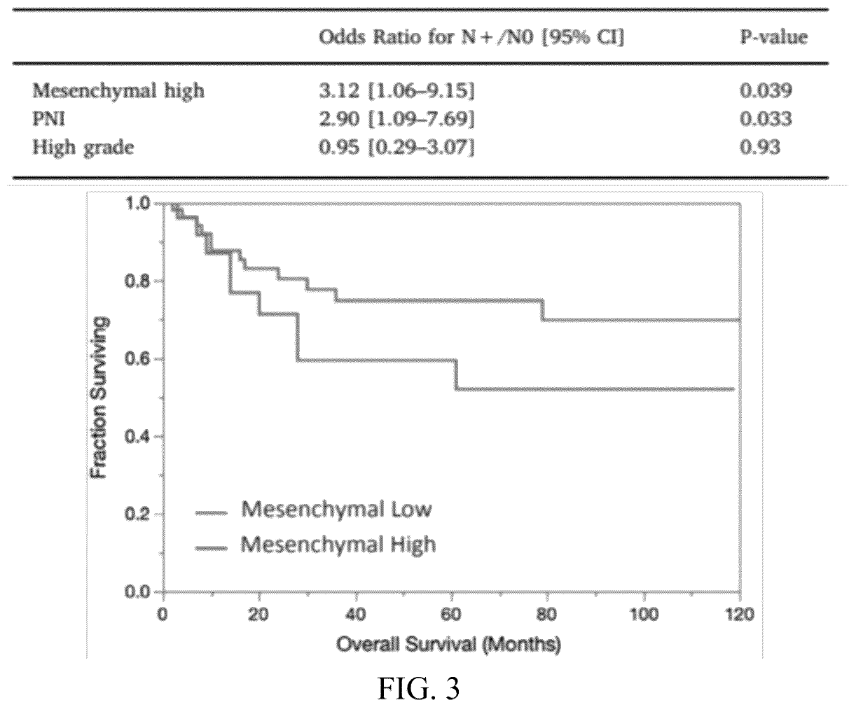

TABLE 1
Six meta-signatures, each derived from multiple related NNMF programs,
(Related to FIG. 3 of Puram, et al. 2017). Genes in each program are
ordered from most to least significant (Puram, et al. 2017).

| Cell Cycle | p-EMT | Epi dif. 1 | Epi dif. 2 | Stress | Hypoxia |
|---|---|---|---|---|---|
| TK1 | SERPINE1 | IL1RN | LY6D | FOS | NDRG1 |
| HMGB2 | TGFBI | SLPI | KRT16 | ATF3 | IGFBP3 |
| ZWINT | MMP10 | CLDN4 | KRT6B | NR4A1 | PTHLH |
| MAD2L1 | LAMC2 | S100A9 | LYPD3 | DUSP1 | EGLN3 |
| TUBA1B | P4HA2 | SPRR1B | KRT6C | ZFP36 | BNIP3 |
| STMN1 | PDPN | PVRL4 | TYMP | PPP1R15A | NDUFA4L2 |
| KIF22 | ITGA5 | RHCG | FABP5 | SGK1 | ERO1L |
| CKS1B | LAMA3 | SDCBP2 | SCO2 | EGR1 | P4HA1 |
| H2AFZ | CDH13 | S100A8 | FGFBP1 | ZC3H12A | SLC2A1 |
| CENPW | TNC | APOBEC3A | JUP | JUNB | ENO2 |
| CDC20 | MMP2 | GRHL1 | IMP4 | FOSB | HK2 |
| DTYMK | EMP3 | SULT2B1 | DSC2 | IER2 | PGF |
| UBE2C | INHBA | ELF3 | TMBIM1 | NFKBIA | LDHA |
| UBE2T | LAMB3 | KRT16 | KRT14 | NFKBIZ | PGK1 |
| NUSAP1 | VIM | PRSS8 | C1QBP | HBEGF | PDK1 |
| RRM2 | SEMA3C | MXD1 | SFN | BTG2 | DHRS3 |
| BIRC5 | PRKCDBP | S100A7 | S100A14 | SOD2 | DDIT4 |
| RNASEH2A | ANXA5 | KRT6B | RAB38 | CDKN1A | PVRL4 |
| PCNA | DHRS7 | LYPD3 | GJB5 | NCOA7 | GPNMB |
| TUBB | ITGB1 | TACSTD2 | MRPL14 | JUN | BIK |
| KPNA2 | ACTN1 | CDKN1A | TRIM29 | MYC | GJB6 |
| ASF1B | CXCR7 | KLK11 | ANXA8L2 | SERTAD1 | C4orf3 |
| TRIP13 | ITGB6 | GPRC5A | KRT6A | CCNL1 | IGFBP2 |
| CCNB1 | IGFBP7 | KLK10 | PDHB | RND3 | FAM162A |
| TPX2 | THBS1 | TMBIM1 | AKR1B10 | PLK2 | GPI |
| CCNB2 | PTHLH | PLAUR | LAD1 | SOCS3 | LPIN3 |
| TYMS | TNFRSF6B | CLDN7 | DSG3 | DNAJB1 | PLAU |
| PTTG1 | PDLIM7 | DUOXA1 | MRPL21 | DUSP2 | ADM |
| KIAA0101 | CAV1 | PDZK1IP1 | NDUFS7 | TSC22D1 | ANGPTL4 |
| GMNN | DKK3 | NCCRP1 | PSMD6 | KLF10 | DARS |
| DNAJC9 | COL17A1 | IDS | AHCY | GADD45B | NUPR1 |
| CCNA2 | LTBP1 | PPL | GBP2 | PMAIP1 | SERPINE1 |
| CKS2 | COL5A2 | ZNF750 | TXN2 | MAFF | PGAM1 |
| MLF1IP | COL1A1 | EMP1 | PSMD13 | ERRFI1 | ALDOA |
| VRK1 | FHL2 | CLDN1 | NOP16 | SLC38A2 | DAAM1 |
| CENPM | TIMP3 | CRB3 | EIF4EBP1 | IRF1 | CXADR |
| PRC1 | PLAU | CYB5R1 | MRPL12 | TOB1 | SEMA4B |
| SPAG5 | LGALS1 | DSC2 | HSD17B10 | ID2 | CA9 |
| TOP2A | PSMD2 | S100P | LGALS7B | KLF6 | CIB1 |
| AURKB | CD63 | GRHL3 | THBD | DNAJA1 | SPRR1B |
| FEN1 | HERPUD1 | SPINT1 | EXOSC4 | TNFAIP3 | PLIN2 |
| TMEM106C | TPM1 | SDR16C5 | APRT | BHLHE40 | WSB1 |
| RRM1 | SLC39A14 | SPRR1A | ANXA8L1 | NXF1 | HILPDA |
| RFC4 | C1S | WBP2 | ATP5G1 | FOSL1 | NOL3 |
| MCM7 | MMP1 | GRB7 | S100A2 | IER3 | PFKFB3 |
| CDKN3 | EXT2 | KLK7 | TBRG4 | DUSP6 | IFNGR1 |
| NUDT1 | COL4A2 | TMEM79 | MAL2 | HCAR2 | H1F0 |
| PBK | PRSS23 | SBSN | NHP2L1 | IL8 | KDM3A |
| MELK | SLC7A8 | PIM1 | DDX39A | CYR61 | BCL6 |
| ANLN | SLC31A2 | CLIC3 | ZNF750 | EFNA1 | BNIP3L |
| MCM5 | ARPC1B | MALAT1 | UBE2L6 | C1R | ZFP36L1 |
| PLK1 | APP | TRIP10 | WDR74 | PHLDA2 | HLA-E |
| GGH | MFAP2 | CAST | PPIF | DNAJB14 | PIK3IP1 |
| MCM4 | MPZL1 | TMPRSS4 | PRMT5 | MCL1 | CLK3 |
| CENPN | DFNA5 | TOM1 | VSNL1 | HERPUD1 | POLR1D |
| TMPO | MT2A | A2ML1 | VPS25 | ADRB2 | BTG1 |
| CDCA3 | MAGED2 | MBOAT2 | SNRNP40 | EIF4A3 | NPC2 |
| DEK | ITGA6 | LGALS3 | ADRM1 | TACSTD2 | LAMP2 |
| RPA2 | FSTL1 | ERO1L | NDUFS8 | ID1 | DSG2 |
| KIF2C | TNFRSF12A | EHF | TUBA1C | ETS2 | SAT1 |
| CDK1 | IL32 | LCN2 | TMEM79 | CD74 | AK4 |
| CDCA5 | COPB2 | YPEL5 | UQCRFS1 | TRIB1 | SMS |
| LSM4 | PTK7 | ALDH3B2 | EIF3K | SLC20A1 | FRMD6 |
| KNSTRN | OCIAD2 | DMKN | NME2 | LOC284454 | CLDND1 |
| TUBG1 | TAX1BP3 | PIK3IP1 | PKP3 | EIF1 | ACP5 |
| SMC4 | SEC13 | CEACAM6 | SERPINB1 | CXCL2 | AP1G2 |
| CSE1L | SERPINH1 | OVOL1 | RPL26L1 | BRD2 | TPI1 |
| UHRF1 | TPM4 | TMPRSS11E | EIF6 | RASD1 | PLAUR |
| RANBP1 | MYH9 | CD55 | DSP | LDLR | BCL10 |
| CDCA8 | ANXA8L1 | KLK6 | PHLDA2 | EGR2 | TMEM59 |
| MCM2 | PLOD2 | SPRR2D | S100A16 | TFRC | HAS3 |
| RFC2 | GALNT2 | NDRG2 | LGALS7 | ADM | SERINC1 |
| HMGN2 | LEPREL1 | CD24 | MT1X | TGIF1 | C1orf43 |
| ATAD2 | MAGED1 | HIST1H1C | UQCRC2 | HLA-DRB1 | ENO1 |
| HAT1 | SLC38A5 | LY6D | EIF3I | OSR2 | CSDA |
| PKMYT1 | FSTL3 | CLIP1 | MRPL24 | SAA1 | PFKP |
| SIVA1 | CD99 | HIST1H2AC | CCT7 | ELF3 | KLHL24 |
| FANCI | F3 | BNIPL | RHOV | CLK1 | HIST1H1C |
| ECT2 | PSAP | QSOX1 | ECE2 | PER2 | RBPJ |
| POLE3 | NMRK1 | ECM1 | SSBP1 | KLF4 | BHLHE40 |
| WDR34 | FKBP9 | DHRS3 | POLDIP2 | GPNMB | GAPDH |
| MCM3 | DSG2 | PPP1R15A | FIS1 | MXD1 | UPK3BL |
| NCAPG2 | ECM1 | TRIM16 | CKMT1A | UBC | LTBP1 |
| TUBB6 | HTRA1 | AQP3 | GJB3 | HLA-DRA | P4HA2 |
| NCAPD2 | SERINC1 | IRF6 | NME1 | SLC3A2 | HBP1 |
| GINS2 | CALU | CSTA | MRPS12 | OVOL1 | GRHL1 |
| TIMELESS | TPST1 | RAB25 | GPS1 | HIST1H2BK | DDIT3 |
| RAD51 | PLOD3 | HOPX | ALG3 | DDX3X | ANXA1 |
| CMC2 | IGFBP3 | GIPC1 | MRPL20 | LAMB3 | ITGA5 |
| OIP5 | FRMD6 | RAB11FIP1 | EMC6 | ZNF622 | LOC100862671 |
| TUBB4B | CXCL14 | CSTB | SRD5A1 | TUBB2A | PLS3 |
| APOBEC3B | SERPINE2 | KRT6C | PA2G4 | ZFAND5 | TSC22D2 |
| ORC6 | RABAC1 | PKP1 | ECSIT | IRF6 | GLTP |
| C19orf48 | TMED9 | JUP | MRPL23 | TNF | PLOD2 |
| SNRNP25 | NAGK | MAFF | NAA20 | BTG1 | PERP |
| RFC3 | BMP1 | DSG3 | HMOX2 | LMNA | MALL |
| TROAP | ESYT1 | AKTIP | COA4 | MAP1LC3B | CTNND1 |
| EBP | STON2 | KLF3 | DCXR | TSC22D3 | KDM5B |
| DKC1 | TAGLN | HSPB8 | PSMD8 | PLK3 | AHNAK2 |
| H2AFV | GJA1 | H1F0 | WBSCR22 | KLHL21 | PNRC1 |

### Inferring Cancer-Cell Specific Expression

In certain embodiments, a tumor sample comprises malignant cells and tumor microenvironment (TME) cells (e.g., immune cells, stromal cells). In certain embodiments, detecting p-EMT includes bulk RNA sequencing of a tumor sample and obtaining a malignant cell expression level. In certain embodiments, all genes that are not expressed by malignant cells are excluded (i.e., genes that are only expressed by the TME). TME expression may be based on single-cell expression data available for head and neck cancer (e.g., Puram et al., 2017). In certain embodiments, cells with Ea (aggregate expression) above 3 are retained (as calculated only over the malignant cells). While this step reduces the influence of TME on bulk expression profiles, it is not sufficient to control for the effect of TME because most genes expressed by malignant cells are also expressed at comparable levels by additional cell types in the TME. A In certain embodiments, this influence can be removed using regression analysis. For each of the cell types (t) (both TME and malignant cells) the average expression of cell type-specific genes can be used to estimate the relative abundance of the cell type (Frₜ) across all bulk tumors. These estimates can then be used for a multiple linear regression seeking to approximate Ex(i,g), the (log-transformed and centered) expression level of gene g in bulk tumor i, by the sum of Frₜ(i), the estimated relative cell type frequencies of tumor i, multiplied by gene-specific and cell type-specific scaling factors Xₜ(g):

$$Ex ( i , g ) = {\sum }_{t ∈ {T}_{g}} ( {Fr}_{t} ( i ) * {X}_{t} ( g ) ) + R ( i , g )$$

T_{g} includes all the cell types for which the average expression of gene g is lower than that of the malignant cells by at most 2-fold; note that this definition includes also the malignant cell as a cell type, which enables the regression to account for purity. This regression defines the scaling factors Xₜ(g) that minimize the sum of squares of the residuals, R(i,g), which reflect the component of expression level that is not accounted by the expression of cell types T_{g} based on the assumption of linear relationship between cell type abundances and total expression level. In certain embodiments, the residuals are defined as the inferred cancer-cell specific expression.

In certain embodiments, deconvoluting bulk gene expression data obtained from a tumor comprises: a) defining, by a processor, the relative frequency of a set of cell types in the tumor from the bulk gene expression data, wherein the frequency of the cell types is determined by cell type specific gene expression, and wherein the set of cell types comprises one or more cell types selected from the group consisting of T cells, fibroblasts, macrophages, mast cells, B/plasma cells, endothelial cells, myocytes and dendritic cells; and b) defining, by a processor, a linear relationship between the frequency of the non-malignant cell types and the expression of a set of genes, wherein the set of genes comprises genes highly expressed by malignant cells and at most two non-malignant cell types, wherein the set of genes are derived from gene expression analysis of single cells in at least one epithelial tumor, and wherein the residual of the linear relationship defines the malignant cell-specific (MCS) expression profile. The epithelial tumor may be HNSCC. The method may further comprise assigning genes to a specific malignant cell sub-type. In other words, a tumor sample is analyzed for types of non-malignant cells within the tumor based on known cell type markers. This is followed by assigning the detected gene expression to the nonmalignant cells. The residual gene expression data is then assigned to the malignant cell specific sub-population (MCS) in the tumor sample.

### Defining Control Expression and Cell and Sample Scores.

In certain embodiments, the method may further comprise determining a p-EMT score, wherein said score is based on expression of a p-EMT signature for the malignant cell-specific (MCS) expression profile. In certain embodiments, cell scores are used in order to evaluate the degree to which individual cells express a certain pre-defined expression program. These are initially based on the average expression of the genes from the pre-defined program in the respective cell: Given an input set of genes (Gⱼ), Applicants define a score, SCⱼ(i), for each cell i, as the average relative expression (Er) of the genes in Gⱼ. However, such initial scores may be confounded by cell complexity, as cells with higher complexity have more genes detected (i.e. less zeros) and consequently would be expected to have higher cell scores for any gene-set. To control for this effect a control gene-set (Gⱼ^{cont}) may be added; a similar cell score can be calculated with the control gene-set and subtracted from the initial cell scores: SCⱼ(i)=average[Er(Gⱼ,i)]−average[Er(Gⱼ^{cont},i)]. The control gene-set may be selected in a way that ensures similar properties (distribution of expression levels) to that of the input gene-set to properly control for the effect of complexity. In certain embodiments, a control level of expression of a pre-defined program (e.g., p-EMT program) is determined across many tumor samples or population of subjects to obtain a control average expression of the pre-defined program (e.g., p-EMT signature genes or polypeptides). Such population may comprise without limitation 2 or more, 10 or more, 100 or more, or even several hundred or more individuals. In certain embodiments, the control level includes an average expression of the p-EMT genes being used (e.g., 4-100 genes) across tumors that are p-EMT high, p-EMT low or have intermediate expression levels. In certain embodiments, the control level is obtained by determining the expression of the genes or polypeptides in more than 3 tumors, more than 10 tumors, or more than 100 tumors. In certain embodiments, the tumors used to obtain the control level have a range of expression for the p-EMT genes from high to low and a median or intermediate expression level is used as the control level. Control expression levels can be obtained from a database of bulk tumor samples or tumor samples previously analyzed. Thus, the control expression can be used as a reference for determining p-EMT high and low tumors. Selecting for control genes with similar average expression as the control expression level enables improved control between tumor samples having different complexity and results in generating a score of zero if the expression of the p-EMT signature is the same as the control expression level. In certain embodiments, all analyzed genes (e.g., p-EMT genes) are binned into 25 bins of equal size based on their aggregate expression levels (Ea). Next, for each gene in the given gene-set, about 20-100 genes are randomly selected from the same expression bin. In this way, the control gene-set has a comparable distribution of expression levels to that of the considered gene-set, and is 20-100-fold larger, such that its average expression is analogous to averaging over 20-100 randomly-selected gene-sets of the same size as the considered gene-set. A similar approach can be used to define bulk sample scores from TCGA.

p-EMT Stratification of Samples

In certain embodiments, the subject is p-EMT high if a p-EMT signature is detected above a p-EMT high reference level as described herein. In certain embodiments, the subject is p-EMT high if a p-EMT signature is detected above a p-EMT high reference level and the epithelial signature is detected below an epithelial low reference. In certain embodiments, sample scores can be defined for all tumors based on the inferred cancer-cell specific expression of the p-EMT and epithelial differentiation (Epi. Diff. 2) signatures; only the subset of genes from these signatures which are included in the inferred cancer-cell specific expression are used for these scores. In certain embodiments, the tumors are ranked based on their p-EMT score minus the epithelial differentiation, and defined the highest 40% as p-EMT high and the lowest 40% as p-EMT low, while excluding the remaining 20% of tumors with intermediate scores.

In certain embodiments, stratification can be specific to the demographic group the subject belongs to. A demographic group may be defined as a subset of the general population based on some factor, such as, the group's age, gender, occupation, nationality, ethnic background, smoking status, etc. In certain embodiments, a p-EMT score is calculated using a control average expression of head and neck cancers specific to the demographic group. Thus, a control expression level can be determined for tumors belonging to a specific demographic group. In certain embodiments, stratification of subjects into p-EMT high and p-EMT low depends on the demographic group. For example, an African American male may be considered p-EMT high with a lower p-EMT score than a subject in another demographic group. In certain embodiments, a lower p-EMT score in a specific demographic still indicates a high risk. In certain embodiments, a p-EMT low score in a specific demographic indicates a low risk. The p-EMT score may be more predictive in specific demographic groups. For example, a higher percentage of a demographic group with a specific score are true low or high risk subjects. For example, if an African American subject has a p-EMT high score there is a higher probability of high risk for death and/or metastasis and if the subject has a low p-EMT score there is a higher probability of low risk for death and/or metastasis as compared to another demographic group. In certain embodiments, a p-EMT high score is greater than 0.5 and a p-EMT low score is less than −0.5 for any demographic selected from the group consisting of Caucasian, non-smoker and female. In certain embodiments, a p-EMT high score is greater than 0.4 and a p-EMT low score is less than −0.4 for non-Caucasians. In certain embodiments, a p-EMT high score is greater than 0.3 and a p-EMT low score is less than −0.3 for males. In certain embodiments, a p-EMT high score is greater than 0.2 and a p-EMT low score is less than −0.2 for African Americans. In certain embodiments, a p-EMT high score is greater than 0.1 and a p-EMT low score is less than −0.1 for African American males. In certain embodiments, the age of a subject increases risk. In certain embodiments, subjects older than 35, 40, 45, 50, 55 or 60 years old have increasing risk as age increases. In certain embodiments, HPV infection increases risk. Smoking at the time of HNSCC diagnosis is associated with lower survival than nonsmoking and patients who were smokers at diagnosis were almost twice as likely to die during a 15 year study period as nonsmokers (see, e.g., Osazuwa-Peters, Nosayaba et al. “Association Between Head and Neck Squamous Cell Carcinoma Survival, Smoking at Diagnosis, and Marital Status.” JAMA otolaryngology—head & neck surgery vol. 144, 1 (2018): 43-50). Thus, in certain embodiments, detection of p-EMT high in a subject at diagnosis makes the subject at least twice as likely to die as a subject that is p-EMT low at diagnosis.

A “deviation” of a p-EMT score from a control score may generally encompass any direction (e.g., increase: first value>second value; or decrease: first value<second value) and any extent of alteration. For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value for a given demographic population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

### Detection, Prognosis and Monitoring Treatment

In certain embodiments, the p-EMT score is used for prognosis or diagnosis of a tumor. In certain embodiments, detection of a high p-EMT score can indicate high risk or low probability of survival. Thus, detection of p-EMT high may dictate intensification of the treatment regimen for the subjects and detection of p-EMT low may dictate de-intensification of the treatment regimen for the subjects (treatments described further herein). In certain embodiments, detection of a high p-EMT score can indicate an occult metastasis in a clinically N0 neck. In certain embodiments, detection of a high p-EMT score can indicate perineural invasion (PNI) in a clinically N0 neck.

In certain embodiments, a p-EMT score is monitored in a subject undergoing treatment. Specific treatments are described further herein, however, certain treatments are able to shift the p-EMT signature from p-EMT high to p-EMT low (see, e.g., an inhibitor of TGF beta signaling). Thus, the efficacy of a treatment can be monitored by detection of p-EMT. In certain embodiments, the p-EMT score is determined at diagnosis to determine a baseline level. Any increase in score over the baseline may indicate that the subject is high risk even if the score is not p-EMT high.

In an embodiment of the invention, p-EMT signatures are useful in monitoring subjects undergoing treatments and therapies for cancer to determine efficaciousness of the treatment or therapy. In an embodiment of the invention, these signatures are useful in monitoring subjects undergoing treatments and therapies for cancer to determine whether the patient is responsive to the treatment or therapy. In an embodiment of the invention, these signatures are also useful for selecting or modifying therapies and treatments that would be efficacious in treating, delaying the progression of or otherwise ameliorating a symptom of cancer. In an embodiment of the invention, the signatures provided herein are used for selecting a group of patients at a specific state of a disease with accuracy that facilitates selection of treatments.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Detection of p-EMT Signature

In certain embodiments, the p-EMT signature is detected in malignant cells or the fraction of expression representing malignant cell expression. In certain embodiments, p-EMT is detected by detecting RNA levels. In certain embodiments, detecting RNA includes RNA-seq, fluorescently bar-coded oligonucleotide probes (see e.g., Geiss G K, et al., Direct multiplexed measurement of gene expression with color-coded probe pairs. Nat Biotechnol. 2008 March; 26(3):317-25), RT-PCR, or hybridization. In certain embodiments, p-EMT is detected by detecting protein levels. In certain embodiments, detecting protein includes western blot, ELISA, mass spectrometry, or immunohistochemistry (IHC). In certain embodiments, the signature genes, biomarkers, and/or cells may be detected or isolated by immunofluorescence, fluorescence activated cell sorting (FACS), mass cytometry (CyTOF), quantitative RT-PCR, single cell qPCR, FISH, RNA-FISH, MERFISH (multiplex (in situ) RNA FISH) and/or by in situ hybridization.

The present invention also may comprise a kit with a detection reagent that binds to one or more biomarkers or can be used to detect one or more biomarkers.

### Sequencing

In certain embodiments, biomarkers are detected by sequencing. In certain embodiments, a target nucleic acid molecule (e.g., RNA molecule), may be sequenced by any method known in the art, for example, methods of high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the present invention involves single cell RNA sequencing (scRNA-seq). In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International Patent Application No. PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International Patent Application No. PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-costRNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Histology

In certain embodiments, histology is used to detect a p-EMT signature. Histology, also known as microscopic anatomy or microanatomy, is the branch of biology which studies the microscopic anatomy of biological tissues. Histology is the microscopic counterpart to gross anatomy, which looks at larger structures visible without a microscope. Although one may divide microscopic anatomy into organology, the study of organs, histology, the study of tissues, and cytology, the study of cells, modern usage places these topics under the field of histology. In medicine, histopathology is the branch of histology that includes the microscopic identification and study of diseased tissue. Biological tissue has little inherent contrast in either the light or electron microscope. Staining is employed to give both contrast to the tissue as well as highlighting particular features of interest. When the stain is used to target a specific chemical component of the tissue (and not the general structure), the term histochemistry is used. Antibodies can be used to specifically visualize proteins, carbohydrates, and lipids. This process is called immunohistochemistry, or when the stain is a fluorescent molecule, immunofluorescence. This technique has greatly increased the ability to identify categories of cells under a microscope. Other advanced techniques, such as nonradioactive in situ hybridization, can be combined with immunochemistry to identify specific DNA or RNA molecules with fluorescent probes or tags that can be used for immunofluorescence and enzyme-linked fluorescence amplification.

### Treatment of Subjects

In certain embodiments, patients suffering from head and neck cancer are differentially treated based on whether the patient is in the high risk group or low risk group as described herein. It will be understood by the skilled person that treating as referred to herein encompasses enhancing treatment, or improving treatment efficacy. Treatment may include tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular cancer. The invention comprehends a treatment method comprising any one of the methods or uses herein discussed.

The phrase “therapeutically effective amount” as used herein refers to a nontoxic but sufficient amount of a drug, agent, or compound to provide a desired therapeutic effect.

As used herein “patient” refers to any human being receiving or who may receive medical treatment.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing a cancer (e.g., a person who is genetically predisposed) may receive prophylactic treatment to inhibit or delay symptoms of the disease.

### TGF Beta Signaling Inhibitors

In certain embodiments, a subject is treated with one or more inhibitors of TGFβ signaling. As described herein, the p-EMT signature may be regulated by TGFβ signaling. Further, inhibitors of TGFβ signaling may shift a tumor from p-EMT high to p-EMT low. In certain embodiments, detection of a p-EMT signature indicates that a therapy targeting the TGFβ pathway should be used in treating cancer. Therapies targeting TGFβ signaling have been described (see e.g., Neuzilleta, et al., Targeting the TGFβ pathway for cancer therapy, Pharmacology & Therapeutics, Volume 147, March 2015, Pages 22-31). In certain embodiments, an epithelial tumor with a high p-EMT score is treated with a known therapy targeting TGFβ signaling. Exemplary inhibitors are provided in Table 2. In certain embodiments, a high p-EMT score may indicate a patient population is more responsive to a therapy targeting TGFβ signaling.

TABLE 2
TGFβ pathway inhibitors in development in cancer.

| | | | |
|---|---|---|---|
| Name | Targets | Trial identifier | Current status |

TGFβ ligand inhibitors

| | | | |
|---|---|---|---|
| Lerdelimumab | TGFβ2 | | Development stopped |
| (CAT-152) | | | |
| Genzyme ® | | | |
| Metelimumab | TGFβ1 | | Development stopped |
| Genzyme ® | | | |
| Fresolimumab | TGFβ1, -β2, -β3 | NCT00356460 | Results in RCC, melanoma, mesothelioma and |
| (GC1008) | | NCT00923169 | glioma; combination phase I/II in progress in |
| Genzyme ®/Aventis ® | | NCT01472731 | breast cancer |
| | | NCT01112293 | |
| | | NCT01401062 | |
| LY2382770 | TGFβ1 | | In progress outside oncology |
| Eli Lilly ® | | | |
| Trabedersen | TGFβ2 | NCT00844064 | Results in glioma, PDAC, CRC, melanoma and |
| (AP12009) | | NCT00431561 | glioblastoma |
| Antisens Pharma ® | | NCT00761280 | |
| Lucanix | TGFβ2 | NCT01058785 | Results in glioma and NSCLC; combination |
| (Belagenpumatucel-L) | | NCT00676507 | phase I in progress |
| NovaRx Corporation ® | | | |
| FANG ™ Vaccine | TGFβ1, -β2 | NCT01061840 | In progress in melanoma, CRC and ovarian |
| (rhGMCSF/shRNAfurin) | | NCT01309230 | cancer |
| Gradalis ® | | NCT01505166 | |
| | | NCT01453361 | |
| Disitertide | TGFβ1 | | In progress outside oncology |
| (P144) | | | |
| Digna Biotech ® | | | |

TGFβ receptor inhibitors

| | | | |
|---|---|---|---|
| Galunisertib | TGFβRI | NCT01246986 | Phase II in progress in PDAC, HCC, glioma and |
| (LY2157299) | | NCT01373164 | glioblastoma |
| Eli Lilly ® | | NCT01220271 | |
| | | NCT02178358 | |
| | | NCT01582269 | |
| TEW-7197 | TGFβRI | NCT02160106 | Phase I in progress |
| MedPacto ® | | | |
| PF-03446962 | ALK-1 | NCT00557856 | Results of phase I; phase II results pending in |
| Pfizer ® | (TGFβRI) | NCT01337050 | HCC and in progress in malignant pleural |
| | | NCT01911273 | mesothelioma and refractory urothelial |
| | | NCT01486368 | carcinoma; combination phase I in progress with |
| | | NCT01620970 | regorafenib in CRC |
| | | NCT02116894 | |
| IMC-TR1 | TGFβRII | NCT01646203 | Phase I in progress |
| (LY3022859) | | | |
| Eli Lilly ® | | | |

CRC: colorectal carcinoma; HCC: hepatocellular carcinoma; NSCLC: non-small cell lung carcinoma; PDAC: pancreatic ductal adenocarcinoma; RCC: Renal cell carcinoma.

### Standard of Care

Aspects of the invention involve modifying the therapy within a standard of care based on the detection of ap-EMT signature as described herein. In one embodiment, therapy comprising an agent is administered within a standard of care where addition of the agent is synergistic within the steps of the standard of care. In one embodiment, the agent targets TGFβ signaling. In one embodiment, the agent inhibits expression or activity of a gene or polypeptide selected from the p-EMT signature. In one embodiment, the agent targets tumor cells expressing a gene or polypeptide selected from the p-EMT signature. The term “standard of care” as used herein refers to the current treatment that is accepted by medical experts as a proper treatment for a certain type of disease and that is widely used by healthcare professionals. Standard of care is also called best practice, standard medical care, and standard therapy. Standards of care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The standards of care for the most common cancers can be found on the website of National Cancer Institute (www.cancer.gov/cancertopics). A treatment clinical trial is a research study meant to help improve current treatments or obtain information on new treatments for patients with cancer. When clinical trials show that a new treatment is better than the standard treatment, the new treatment may be considered the new standard treatment.

The term “Adjuvant therapy” as used herein refers to any treatment given after primary therapy to increase the chance of long-term disease-free survival. The term “Neoadjuvant therapy” as used herein refers to any treatment given before primary therapy. The term “Primary therapy” as used herein refers to the main treatment used to reduce or eliminate the cancer.

In exemplary embodiments, two types of standard treatment are used to treat HNSCC. In certain embodiments, the standard treatment is surgery or radiation therapy.

Surgery may include neck dissection. In certain embodiments, the current standard of care cannot predict whether a tumor has spread to the lymph nodes and unnecessary neck dissections may be performed. In certain embodiments, only after performing a neck dissection and examination of the dissected tissue can it be determined that the dissection was necessary. In preferred embodiments, neck dissection is used when a p-EMT signature, preferably a p-EMT high signature (or high risk patients), as described herein is detected in a sample obtained from a subject in need thereof. The sample is preferably from a primary tumor. Neck dissection may be delayed when a p-EMT signature is not detected (or low risk patients). In certain embodiments, unnecessary neck dissections may be avoided by incorporating the methods and gene signatures described herein into the standard of care. It will be appreciated by one of ordinary skill in the art that avoiding unnecessary aggressive interventions such as neck dissection also avoids the related potential co-morbidities and mortality associated with such procedures. The invention thus provides a substantial improvement in care of such patients.

There are different types of neck dissection based on the amount of tissue that is removed. Radical neck dissection may comprise surgery to remove tissues in one or both sides of the neck between the jawbone and the collarbone, including the following: 1) all lymph nodes, 2) the jugular vein, and 3) the muscles and nerves that are used for face, neck, and shoulder movement, speech, and swallowing. In most cases, radical neck dissection is used when cancer has spread widely in the neck. However, detection of cancer in the lymph nodes and detection of a p-EMT high signature may indicate that radical neck dissection is required. Modified radical neck dissection may comprise surgery to remove all the lymph nodes in one or both sides of the neck without removing the neck muscles. The nerves and/or the jugular vein may be removed. Partial neck dissection may comprise surgery to remove some of the lymph nodes in the neck. This is also called selective neck dissection. In certain embodiments, radical neck dissection, modified radical neck dissection, or partial neck dissection is used when a p-EMT signature as described herein is detected in a sample obtained from a subject in need thereof. In preferred embodiments, the sample is obtained from a primary tumor. In certain embodiments, detection of a p-EMT signature indicates that a partial neck dissection should be performed due to the high correlation to negative outcomes (e.g., metastasis) and absence of a p-EMT signature indicates that surgery may be delayed. In preferred embodiments, partial neck dissection is used when a p-EMT signature as described herein is detected in a sample obtained from a subject in need thereof. In other preferred embodiments, radical neck dissection or modified radical neck dissection is used instead of partial neck dissection when a p-EMT signature as described herein is detected in a sample obtained from a subject in need thereof. Not being bound by a theory, detection of a p-EMT signature indicates that the more aggressive choice of surgery should be selected. In certain embodiments, the type of neck dissection is performed based on the detection of a p-EMT signature. Not being bound by a theory, if the standard of care indicates a choice between an aggressive surgery and a less aggressive surgery, detection or lack of detection of a p-EMT signature may inform the choice between two options.

In certain embodiments, if a physician removes all of the cancer from a patient that can be seen at the time of surgery, some patients may be given radiation therapy after surgery to destroy any remaining cancer cells. Treatment given after surgery, to lower the risk that the cancer will come back, is called adjuvant therapy. Adjuvant therapy may comprise radiation or chemotherapy. In certain embodiments, detection of a p-EMT signature indicates that adjuvant therapy should be given and absence of a p-EMT signature indicates that further treatment may be delayed or reduced.

As used herein the term “radiation therapy” refers to a cancer treatment that uses high-energy x-rays or other types of radiation to kill cancer cells or keep them from growing. There are two types of radiation therapy. External radiation therapy uses a machine outside the body to send radiation toward the cancer. Certain ways of giving external radiation therapy can help keep radiation from damaging nearby healthy tissue. Intensity-modulated radiation therapy (IMRT) is a type of 3-dimensional (3-D) radiation therapy that uses a computer to make pictures of the size and shape of the tumor. Thin beams of radiation of different intensities (strengths) are aimed at the tumor from many angles. This type of radiation therapy is less likely to cause dry mouth, trouble swallowing, and damage to the skin. Intensity-modulated radiation therapy (EIRT) has become a standard technique for head and neck radiation therapy. EIRT allows a dose-painting technique also known as a simultaneous-integrated-boost (SIB) technique with a dose per fraction slightly higher than 2 Gy, which allows slight shortening of overall treatment time and increases the biologically equivalent dose to the tumor. Internal radiation therapy uses a radioactive substance sealed in needles, seeds, wires, or catheters that are placed directly into or near the cancer. In certain embodiments, an aggressive radiation therapy is used to treat HNSCC where a p-EMT signature is detected.

In certain embodiments, detection of a p-EMT signature is used to determine whether hyperfractionated radiation therapy is used. Hyperfractionated radiation therapy is a type of external radiation treatment in which a smaller than usual total daily dose of radiation is divided into two doses and the treatments are given twice a day. Hyperfractionated radiation therapy is given over the same period of time (days or weeks) as standard radiation therapy.

In addition to surgery and radiation, in certain embodiments detection of a p-EMT signature is used to determine whether chemotherapy should be administered. Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into, e.g., the cerebrospinal fluid, an organ, or a body cavity such as the abdomen, the drugs mainly affect cancer cells in those areas (regional chemotherapy).

Treatment of HNSCC may include radiation therapy, surgery, radiation therapy followed by surgery, chemotherapy followed by radiation therapy, or chemotherapy given at the same time as hyperfractionated radiation therapy. Not being bound by a theory, radiation alone is the least aggressive treatment option, followed by surgery, radiation therapy followed by surgery, chemotherapy followed by radiation therapy, or chemotherapy given at the same time as hyperfractionated radiation therapy. Not being bound by a theory, detection of a p-EMT signature can guide the aggressiveness of a treatment to be administered to a subject in need thereof. In certain embodiments, combined-modality treatment is considered more aggressive treatment. When used in conjunction with surgery, radiation therapy is typically administered postoperatively, postoperative radiation treatment (PORT). Alternative strategies using neoadjuvant chemotherapy and radiation therapy may increase the chance for local control in selected advanced presentations to a level approaching that of resection and PORT. Neoadjuvant chemotherapy as given in clinical trials has been used to shrink tumors and render them more definitively treatable with either surgery or radiation. Chemotherapy is given before the other modalities, hence the designation, neoadjuvant, to distinguish it from standard adjuvant therapy, which is given after or during definitive therapy with radiation or after surgery. Many drug combinations have been used in neoadjuvant chemotherapy. Neoadjuvant chemotherapy is commonly used to treat patients who present with advanced disease to improve locoregional control or survival.

For locally advanced disease, concurrent chemoradiation approaches are superior to radiation therapy alone (Denis, et al., Final results of the 94-01 French Head and Neck Oncology and Radiotherapy Group randomized trial comparing radiotherapy alone with concomitant radiochemotherapy in advanced-stage oropharynx carcinoma. J Clin Oncol 22 (1): 69-76, 2004). This treatment approach emphasizes organ preservation and functionality.

Depending on pathological findings after primary surgery, PORT or postoperative chemoradiation is used in the adjuvant setting for the following histological findings including: T4 disease, Perineural invasion, Lymphovascular invasion, Positive margins or margins less than 5 mm, Extracapsular extension of a lymph node, Two or more involved lymph nodes. In certain embodiments, pathological findings may be combined with detection of a p-EMT signature to a treat a patient in need thereof with postoperative chemoradiation.

The benefit for overall survival has been demonstrated with postoperative chemoradiation therapy using cisplatin; an overall survival benefit has also been found for positive margins and extracapsular extension (Bernier J, et al.: Defining risk levels in locally advanced head and neck cancers: a comparative analysis of concurrent postoperative radiation plus chemotherapy trials of the EORTC (#22931) and RTOG (#9501). Head Neck 27 (10): 843-50, 2005; Cooper J S, et al.: Long-term follow-up of the RTOG 9501/intergroup phase III trial: postoperative concurrent radiation therapy and chemotherapy in high-risk squamous cell carcinoma of the head and neck. Int J Radiat Oncol Biol Phys 84 (5): 1198-205, 2012; Cooper J S, et al.: Postoperative concurrent radiotherapy and chemotherapy for high-risk squamous-cell carcinoma of the head and neck. N Engl J Med 350 (19): 1937-44, 2004; and Bernier J, et al.: Postoperative irradiation with or without concomitant chemotherapy for locally advanced head and neck cancer. N Engl J Med 350 (19): 1945-52, 2004). Not being bound by a theory, detection of a p-EMT signature may be used to select candidates for postoperative chemoradiation therapy.

The present invention advantageously provides a p-EMT signature that positively correlates with the histological features of HNSCC and can be used to predict negative pathological features (e.g., extracapsular extension and lymphovascular invasion), which are clear indications for administering chemoradiation to a surgical intervention. Thus, the signature can predict which patients need chemotherapy and radiation and in some cases this may affect the decision to perform surgery in the first place. In one embodiment, surgery may not be performed and a patient may be first treated with a chemoradiation regimen.

In a randomized trial of locally advanced head and neck cancer patients, curative-intent radiation therapy alone (213 patients) was compared with radiation therapy plus weekly cetuximab (211 patients) (Bonner J A, Harari P M, Giralt J, et al.: Radiotherapy plus cetuximab for squamous-cell carcinoma of the head and neck. N Engl J Med 354 (6): 567-78, 2006). Cetuximab is an epidermal growth factor receptor (EGFR) inhibitor used for the treatment of metastatic colorectal cancer, metastatic non-small cell lung cancer and head and neck cancer. Cetuximab is a chimeric (mouse/human) monoclonal antibody given by intravenous infusion. The initial dose was 400 mg per square meter of body-surface area 1 week before starting radiation therapy followed by 250 mg per square meter weekly for the duration of the radiation therapy. At a median follow up of 54 months, patients treated with cetuximab and radiation therapy demonstrated significantly higher progression-free survival (hazard ratio for disease progression or death, 0.70; P=0.006). Patients in the cetuximab arm experienced higher rates of acneiform rash and infusion reactions, although the incidence of other grade 3 or higher toxicities, including mucositis, did not differ significantly between the two groups. In certain embodiments, radiation therapy plus weekly cetuximab may be administered before metastasis or locally advanced cancer is detected in patients positive for a p-EMT signature.

Aspects of the invention involve targeting proliferating cell types. In certain embodiments, targeting reduces the viability or reduces the invasiveness of p-EMT high cells comprised by the epithelial tumor. In one embodiment, the cells are killed or removed by targeting. In another embodiment, the cells no longer express a p-EMT signature. In certain embodiments, reducing the activity or inhibiting the expression of a p-EMT signature gene may cause loss of the p-EMT signature and improve prognosis. Targeting may be by use of small molecules, antibodies, antibody fragments, antibody like platforms and antibody drug conjugates. Targeting agents may include, but are not limited to, single-chain immunotoxins reactive with human epithelial tumor cells. Antibody drug conjugates are well known in the art.

### Immunotherapy

In certain embodiments, an immunotherapy is administered to a subject. In certain embodiments, the immunotherapy is a checkpoint blockade therapy (CPB). In certain embodiments, the immunotherapy is adoptive cell transfer (ACT).

The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014; 9,320,811; and 9,062,111. Specific check point inhibitors include, but are not limited to, anti-CTLA4 antibodies (e.g., Ipilimumab and Tremelimumab), anti-PD-1 antibodies (e.g., Nivolumab, Pembrolizumab), and anti-PD-L1 antibodies (e.g., Atezolizumab).

In certain embodiments, checkpoint inhibition may be enhanced by administering a TLR agonist to enhance anti-tumor immunity (see, e.g., Urban-Wojciuk, et al., The Role of TLRs in Anti-cancer Immunity and Tumor Rejection, Front Immunol. 2019; 10: 2388; and Kaczanowska et al., TLR agonists: our best frenemy in cancer immunotherapy, J Leukoc Biol. 2013 June; 93(6): 847-863). In certain embodiments, a TLR9 agonist is administered (see, e.g., Chuang, et al., Adjuvant Effect of Toll-Like Receptor 9 Activation on Cancer Immunotherapy Using Checkpoint Blockade, Front. Immunol., 29 May 2020; and Reilley, et al., TLR9 activation cooperates with T cell checkpoint blockade to regress poorly immunogenic melanoma, J. Immunotherapy Cancer, 2019, 7, 323). In certain embodiments, TLR agonists are delivered in a nanoparticle system (see, e.g., Buss and Bhatia, Nanoparticle delivery of immunostimulatory oligonucleotides enhances response to checkpoint inhibitor therapeutics, Proc Natl Acad Sci USA. 2020 Jun. 3; 202001569).

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

Agents Targeting Malignant Cells Expressing a p-EMT Signature

In certain embodiments, an agent targets one or more p-EMT signature genes or polypeptides. In certain embodiments, may be an antibody, antibody fragment, intrabody, antibody-like protein scaffold, aptamer, polypeptide, small molecule, small molecule degrader, genetic modifying agent, or any combination thereof.

### Antibodies

In certain embodiments, an agent that targets one or more p-EMT signature genes or polypeptides is an antibody. In certain embodiments, an antibody targets one or more surface p-EMT signature genes or polypeptides. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by p pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}I domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}—Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Bispecific Antibodies

In certain embodiments, bispecific antibodies are used to target the p-EMT high malignant cells. In certain embodiments, bispecific antibodies are used to target immune cells to the p-EMT high malignant cells. Bi-specific antigen-binding constructs, e.g., bi-specific antibodies (bsAb) or BiTEs, bind two antigens (see, e.g., Suurs et al., A review of bispecific antibodies and antibody constructs in oncology and clinical challenges. Pharmacol Ther. 2019 September; 201:103-119; and Huehls, et al., Bispecific T cell engagers for cancer immunotherapy. Immunol Cell Biol. 2015 March; 93(3): 290-296). The bi-specific antigen-binding construct includes two antigen-binding polypeptide constructs, e.g., antigen binding domains, wherein at least one polypeptide construct specifically binds to a tumor surface protein. In some embodiments, the antigen-binding construct is derived from known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

In certain embodiments, immune cells can be engaged to tumor cells. In certain embodiments, tumor cells are targeted with a bsAb having affinity for both the tumor and a payload. In certain embodiments, two targets are disrupted on a tumor cell by the bsAb (e.g., any two p-EMT genes or polypeptides). By means of an example, an agent, such as a bi-specific antibody, capable of specifically binding to a gene product expressed on the cell surface of the immune cells (e.g., CD3, CD8, CD28, CD16) and a tumor cell (e.g., p-EMT high) may be used for targeting polyfunctional immune cells to tumor cells. Immune cells targeted to a tumor may include T cells or Natural Killer cells.

### Antibody Drug Conjugates

In certain embodiments, antibody drug conjugates (ADC) target p-EMT malignant cells with one or more drugs. The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8.

In certain embodiments, the ADC specifically binds to a gene product expressed on the cell surface of a tumor cell. By means of an example, an agent, such as an antibody, capable of specifically binding to a gene product expressed on the cell surface of the tumor cells may be conjugated with a therapeutic or effector agent for targeted delivery of the therapeutic or effector agent to the immune cells.

Examples of such therapeutic or effector agents include immunomodulatory classes as discussed herein, such as without limitation a toxin, drug, radionuclide, cytokine, lymphokine, chemokine, growth factor, tumor necrosis factor, hormone, hormone antagonist, enzyme, oligonucleotide, siRNA, RNAi, photoactive therapeutic agent, anti-angiogenic agent and pro-apoptotic agent.

Non-limiting examples of drugs that may be included in the ADCs are mitotic inhibitors (e.g., maytansinoid DM4), antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

Example toxins include ricin, abrin, alpha toxin, saporin, ribonuclease (RNase), DNase I, Staphylococcal enterotoxin-A, pokeweed antiviral protein, gelonin, diphtheria toxin, Pseudomonas exotoxin, or Pseudomonas endotoxin.

Example radionuclides include ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo or ^{99m}Tc. Preferably, the radionuclide may be an alpha-particle-emitting radionuclide.

Example enzymes include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase or acetylcholinesterase. Such enzymes may be used, for example, in combination with prodrugs that are administered in relatively non-toxic form and converted at the target site by the enzyme into a cytotoxic agent. In other alternatives, a drug may be converted into less toxic form by endogenous enzymes in the subject but may be reconverted into a cytotoxic form by the therapeutic enzyme.

### Aptamers

Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH), 2′-fluoro (2′-F), and/or 2′-0-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein.

### Small Molecules

In certain embodiments, an agent that targets one or more p-EMT signature genes or polypeptides is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins as described herein.

### Genetic Modifying Agents

In certain embodiments, an agent that targets one or more p-EMT signature genes or polypeptides is a genetic modifying agent. In certain embodiments, the genetic modifying agent comprises a CRISPR system, RNAi system, a zinc finger nuclease system, a TALE, or a meganuclease. In certain embodiments, the CRISPR system comprises a CRISPR-Cas base editing system, a prime editor system, or a CAST system.

### CRISPR-Cas Modification

In some embodiments, a polynucleotide of the present invention described elsewhere herein can be modified using a CRISPR-Cas and/or Cas-based system (e.g., genomic DNA or mRNA, preferably, for a disease gene). The nucleotide sequence may be or encode one or more components of a CRISPR-Cas system. For example, the nucleotide sequences may be or encode guide RNAs. The nucleotide sequences may also encode CRISPR proteins, variants thereof, or fragments thereof.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

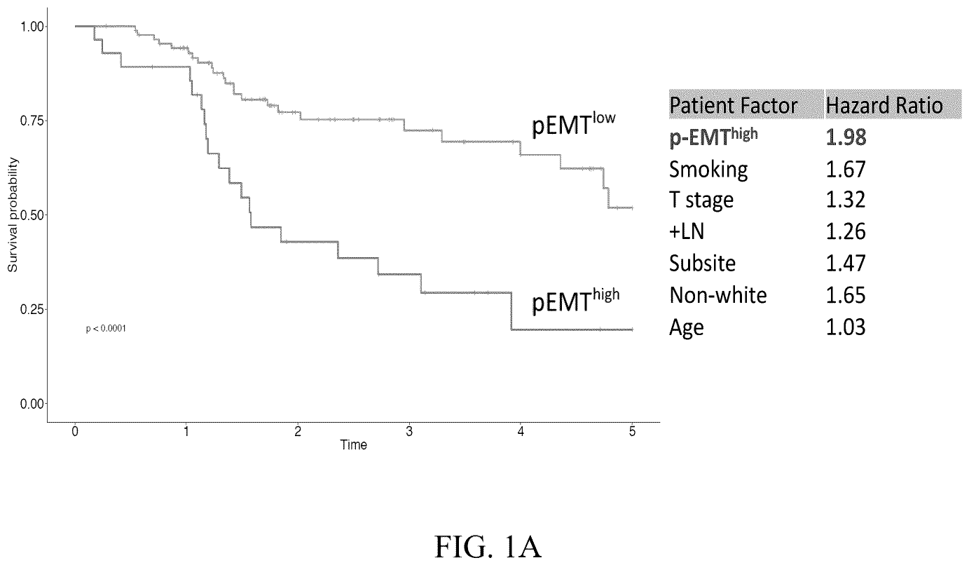

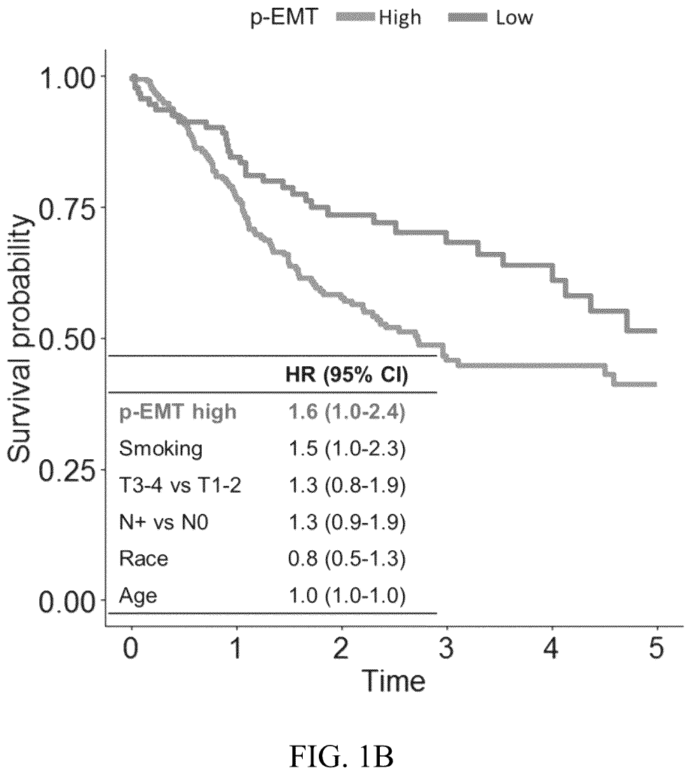

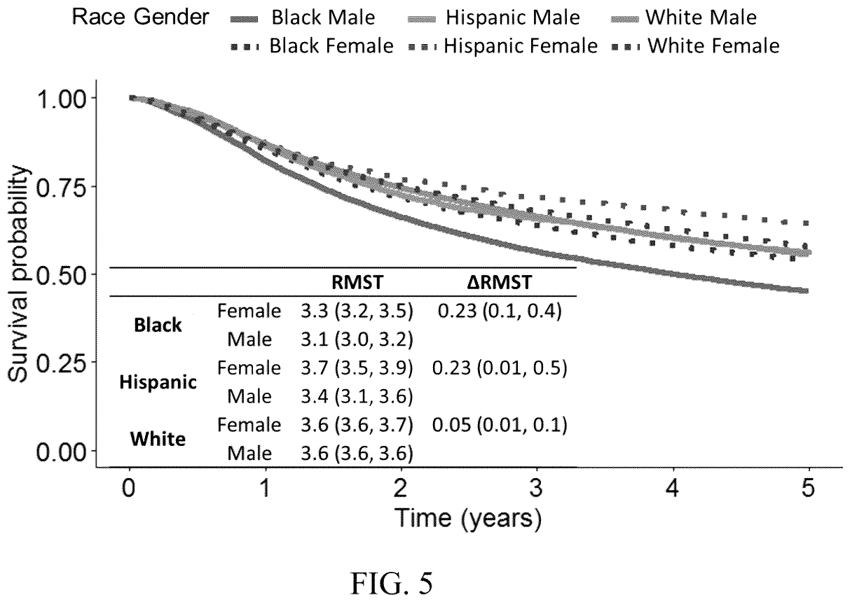

In some embodiments, the CRISPR-Cas system that can be used to modify a polynucleotide of the present invention described herein can be a Class 1 CRISPR-Cas system. Class 1 CRISPR-Cas systems are divided into Types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

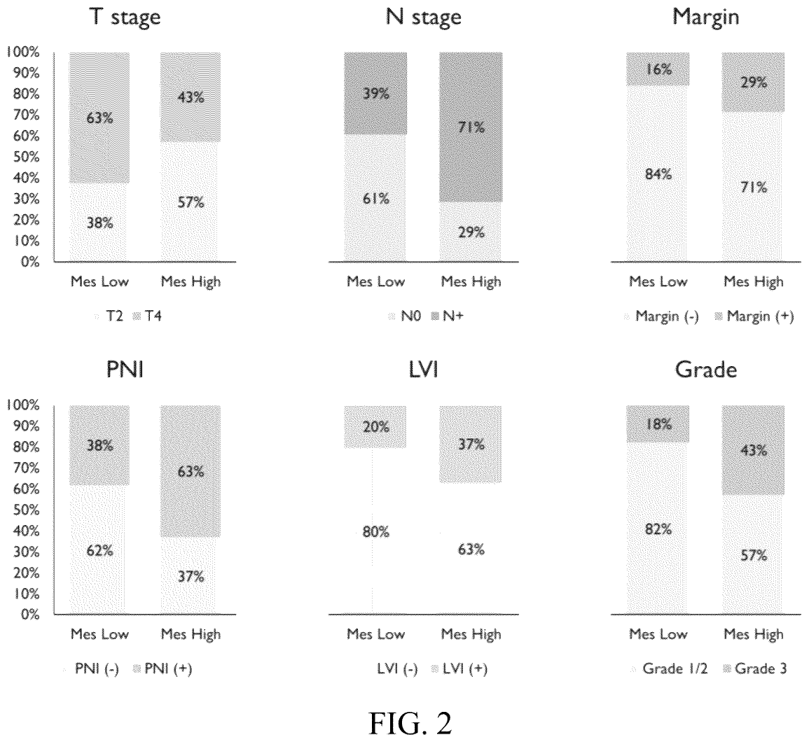

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system (See e.g. Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRISPR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

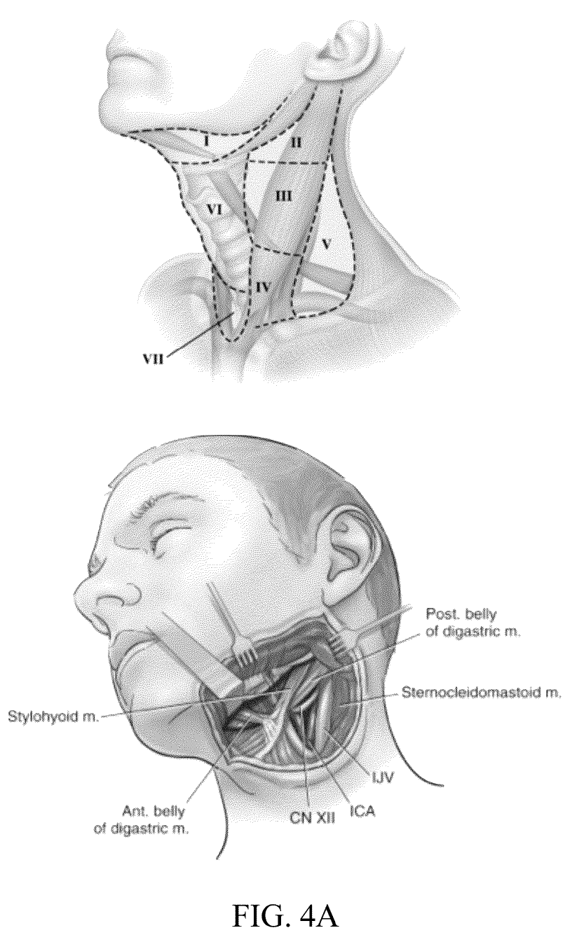

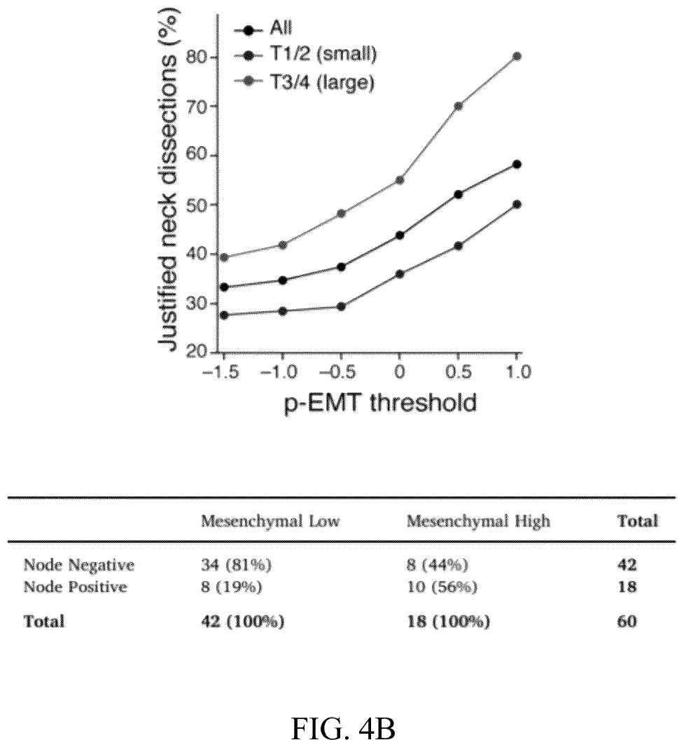

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 below shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein His A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 1)
M D P I R S R T P S P A R E L L S G P Q P D G V Q P T A D R G V S P P A G G P
L D G L P A R R T M S R T R L P S P P A P S P A F S A D S F S D L L R Q F D P S L F N T S
L F D S L P P F G A H H T E A A T G E W D E V Q S G L R A A D A P P P T M R V A V T A
A R P P R A K P A P R R R A A Q P S D A S P A A Q V D L R T L G Y S Q Q Q Q E K I K P
K V R S T V A Q H H E A L V G H G F T H A H I V A L S Q H P A A L G T V A V K Y Q D
M I A A L P E A T H E A I V G V G K Q W S G A R A L E A L L T V A G E L R G P P L Q L
D T G Q L L K I A K R G G V T A V E A V H A W R N A L T G A P L N

An exemplary amino acid sequence of a C-terminal capping region is:

(SEQ ID NO: 2)
R P A L E S I V A Q L S R P D P A L A A L T N D H L V A L A C L G G R P A L
D A V K K G L P H A P A L I K R T N R R I P E R T S H R V A D H A Q V V R V L G F F Q
C H S H P A Q A F D D A M T Q F G M S R H G L L Q L F R R V G V T E L E A R S G T L P
P A S Q R W D R I L Q A S G M K R A K P S P T S T Q T P D Q A S L H A F A D S L E R D
L D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 3) or PKKKRKVEAS (SEQ ID NO: 4); the NLS from nucleoplasmin (e.g., the nucleoplasmin bipartite NLS with the sequence KRPAATKKAGQAKKKK (SEQ ID NO: 5)); the c-myc NLS having the amino acid sequence PAAKRVKLD (SEQ ID NO: 6) or RQRRNELKRSP (SEQ ID NO: 7); the hRNPA1 M9 NLS having the sequence NQSSNFGPMKGGNFGGRSSGPYGGGGQYFAKPRNQGGY (SEQ ID NO: 8); the sequence RMRIZFKNKGKDTAELRRRRVEVSVELRKAKKDEQILKRRNV (SEQ ID NO: 9) of the IBB domain from importin-alpha; the sequences VSRKRPRP (SEQ ID NO: 10) and PPKKARED (SEQ ID NO: 11) of the myoma T protein; the sequence PQPKKKPL (SEQ ID NO: 12) of human p53; the sequence SALIKKKKKMAP (SEQ ID NO: 13) of mouse c-abl IV; the sequences DRLRR (SEQ ID NO: 14) and PKQKKRK (SEQ ID NO: 15) of the influenza virus NS1; the sequence RKLKKKIKKL (SEQ ID NO: 16) of the Hepatitis virus delta antigen; the sequence REKKKFLKRR (SEQ ID NO: 17) of the mouse Mxl protein; the sequence KRKGDEVDGVDEVAKKKSKK (SEQ ID NO: 18) of the human poly(ADP-ribose) polymerase; and the sequence RKCLQAGMNLEARKTKK (SEQ ID NO: 19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA”, used interchangeably herein, are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Administration

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease (e.g., metastatic disease). The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

### Screening for Modulating Agents

A further aspect of the invention relates to a method for identifying an agent capable of modulating or shifting a p-EMT signature as disclosed herein, comprising: a) applying a candidate agent to a cell or population of cells having a p-EMT signature; b) detecting modulation of the p-EMT signature for the cell or cell population by the candidate agent, thereby identifying the agent. In certain embodiments, steps can include administering candidate modulating agents to cells, detecting identified cell (sub)populations for changes in signatures, or identifying relative changes in cell (sub) populations which may comprise detecting relative abundance of particular gene signatures. Screening can be performed in vitro (e.g., tissue culture) or in vivo (e.g., a tumor model).

The term “modulate” or “shift” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase or decrease in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place. Agents may include any potential class of biologically active conditions, substances or agents, such as for instance antibodies, proteins, peptides, nucleic acids, oligonucleotides, small molecules, or combinations thereof, as described herein. The methods of screening can be utilized for screening of chemical libraries. For example, a population of cells can be exposed to a chemical (for example a therapeutic agent or potential therapeutic agent) and the like. After an agent is applied, a representative sample can be subjected to analysis, for example at various time points, and compared to a control, such as a sample of untreated cells or a standard value.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The p-EMT signature of the present invention may be used to screen for drugs that reduce the signature in cells as described herein. The signature may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes, and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a p-EMT signature of the present invention in silico.

Further embodiments are illustrated in the following Examples which are given for illustrative purposes only and are not intended to limit the scope of the invention.

### EXAMPLES

### Example 1—the p-EMT Pathway is a Better Predictor of OCSCC Outcome than Smoking History

To investigate how intra-tumoral heterogeneity contributes to patient outcomes, Applicants previously characterized OCSCC expression heterogeneity at a single cell resolution. Applicants used scRNA-seq (SMART-seq2) to profile ˜6,000 cells from primary OCSCC and matched lymph node (LN) metastases (Puram et al., 2017). Applicants separated malignant and non-malignant cells using 1) expression of epithelial markers (e.g., cytokeratins) and 2) presence or absence of copy number variations (CNVs). Applicants then broadly clustered malignant and non-malignant cells using multidimensional, t-distributed stochastic neighbor embedding analysis (t-SNE). These analyses showed that while non-malignant cells clustered across tumors, malignant cells clustered almost exclusively by their tumor of origin. For malignant cells, Applicants clustered profiles from individual tumors to reveal subpopulations related to the cell cycle, epithelial differentiation, stress, hypoxia, and an EMT-like program, which has some features of EMT (e.g., VIM, ITGA5), yet retains expression of epithelial markers and expresses Snail2, but not other classical EMT TFs. Applicants, therefore, considered this latter program apartial EMT (p-EMT) program. When this analysis was applied to all tumors, Applicants found these programs were remarkably consistent (Puram et al., 2017), suggesting sub-populations were robust.

Since EMT has a well-established role in invasion, Applicants sought to understand how the p-EMT state might contribute to tumorigenic phenotypes in OCSCC. Applicants analyzed the large bulk RNA-seq repository from the Cancer Genome Atlas (TCGA) and deconvolved these data using the single cell profiles to correlate disease progression with p-EMT. Applicants found that tumors with multiple LNs significantly upregulated p-EMT genes relative to tumors without metastases, suggesting that the p-EMT state is associated with the spread of the disease. To further explore this hypothesis, Applicants correlated clinical annotations in TCGA with p-EMT expression. Applicants found that p-EMT was significantly associated with the presence of LN metastases, multiple LN metastases, advanced nodal stage, higher grade, extracapsular extension (ECE), and lymphovascular invasion (LVI). To experimentally validate these associations, Applicants sorted two OCSCC cell lines (SCC9, Cal27) into p-EMT high and low subpopulations based on the expression of surface markers identified in the single cell analysis. Applicants then assessed for invasion using a Matrigel assay. Applicants found that the p-EMT population was significantly more invasive than control (unsorted) cells, while the non-p-EMT cells were less invasive. In other analyses, Applicants determined that among patients with clinically node-negative disease (cN0), high p-EMT was strongly predictive of occult nodal disease ultimately found on surgical pathology (Puram et al., 2017). Together, these results uncovered a striking association between the p-EMT state and adverse histopathologic features, including metastasis in OCSCC.

In further analyses, staining for p-EMT markers revealed localization of p-EMT at the leading edge of tumors, in contrast to the program of epithelial differentiation (see, e.g., WO2018191553A1, FIG. 4A-C). In further analyses, in a small cohort of OCSCC patients, Applicants found that p-EMT score as determined by LAMC2, LAMB3, and PDPN is higher in patients with nodal metastasis, LVI, and PNI compared to patients without these features (Parikh, Puram, et al., Oral Oncology, 2019). These data highlight the potential of IHC markers as a surrogate for p-EMT and its adverse histopathologic features; however, these analyses were limited to a small cohort of predominantly white patients.

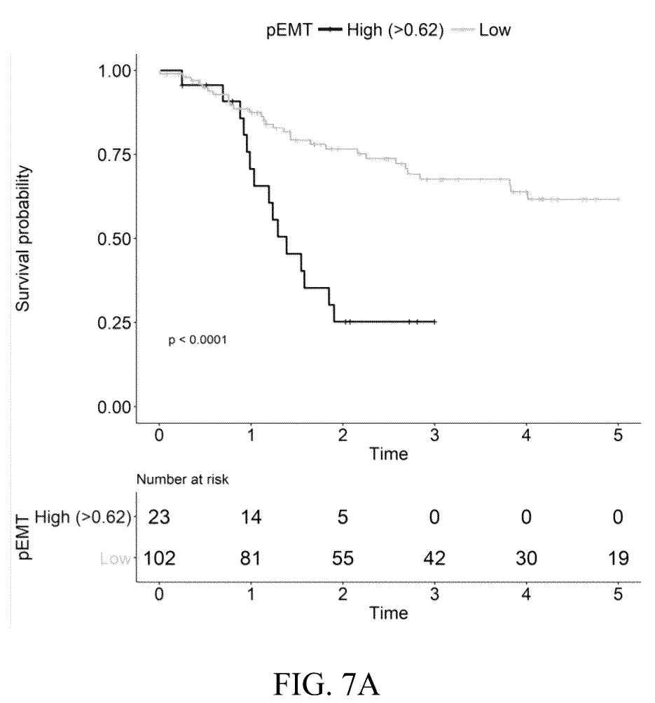

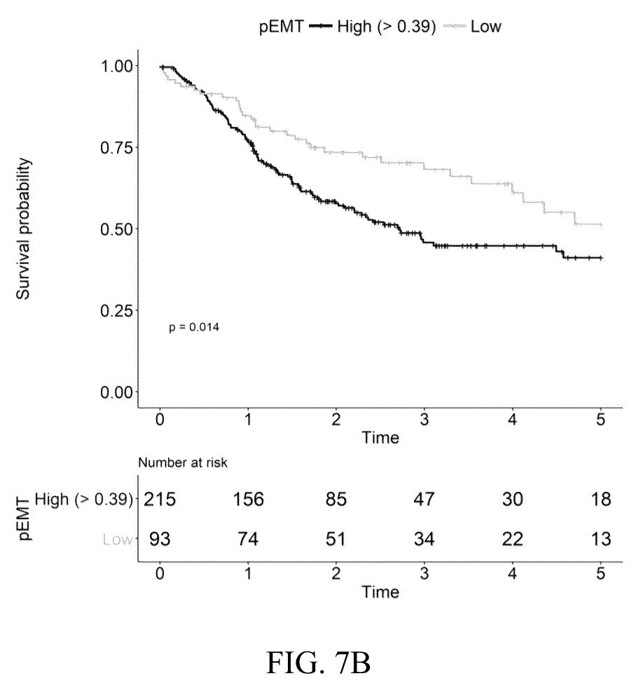

To investigate how intra-tumoral heterogeneity contributes to patient outcomes, Applicants utilized the deconvolved p-EMT scores from OCSCC TCGA tumors and examined survival. Applicants then performed a rigorous comparison of inferred p-EMT scores to other standard histopathological features in multivariable analysis of survival in these tumors adjusting for age, gender, race, and stage (FIG. 1A-B). Remarkably, despite adjusting for tumor size (T stage), metastasis (N stage), and other available socioeconomic data, p-EMT appeared to have an independent association with survival in OCSCC. Perhaps most strikingly, Applicants found that the inferred p-EMT scores were more predictive of disease outcome in OCSCC than even well-established factors such as smoking. Additionally, clinical features are different in p-EMT low and p-EMT high tumors (FIG. 2). For example, 63% of p-EMT high tumors have PNI and 71% are node positive (N+). Additionally, p-EMT high expression predicts N+ better than PNI and tumor grade (FIG. 3). Additionally, justified neck dissections increases with p-EMT score (FIG. 4A-B). Detection of p-EMT low predicts node negative tumors in 81% of p-EMT low tumors and 56% of p-EMT high tumors are node positive tumors (FIG. 4B). Further p-EMT shows a large difference in survival for both larynx and oral cavity cancer (FIG. 7A-B). Thus, detection of p-EMT high dictates intensification of the treatment regimen for the subjects and detection of p-EMT low dictates de-intensification of the treatment regimen for the subjects.

### Example 2—Sociodemographics Influence the Biology of OCSCC Tumors

Disparities in cancer are not solely attributable to the environment and social determinants of health but may also reflect the complex interplay between the genomic landscape and environmental factors. In colorectal cancer, driver mutations in EPHA6 and FLCN have been found exclusively in African Americans. Additionally, racial differences in DNA methylation were identified in both normal and malignant tissue. Similarly, triple-negative breast cancer—the most aggressive subtype—is far more common among African Americans, contributing to racial disparities. In OCSCC, there has been limited research into molecular differences by sociodemographics. TCGA has been the primary source of genomic data for OCSCC research, but is severely limited by the low number of African Americans and lack of information on socioeconomic status. One recent study found that African American larynx and oral cavity tumors are more likely to have overexpression of the POL3 expression quantitative trait locus (eQTL) related to DNA repair and an increased risk of recurrence and death. However, race is only one metric of sociodemographics, and very few studies have investigated the relationship between other metrics of socioeconomic status and genomic markers. Given the limited number of African American cases in publicly available datasets such as TCGA, there is a critical need to characterize the underlying genomic characteristics of socioeconomically-deprived oral cavity cancers and other head and neck cancers.

Gender and race interact to influence survival in head and neck cancer. In general, female OCSCC patients have better survival than males. However, studies focused solely on gender overlook a critical interaction with race (FIG. 5). For example, black males have far worse overall survival and the mean survival time is 2.8 months shorter for minority males (FIG. 5). Applicants recently found that this disparity is more pronounced among Black Americans than either Hispanic Americans or White Americans. These findings suggest that head and neck cancer has large disparities that have further widened as treatment approaches have improved. Indeed, studies have shown that molecular testing is unequal among sociodemographic groups. Even among those that get tested, minorities and underserved populations do not get the recommended precision treatment. Thus, when developing biomarkers for clinical prognostication, important considerations must be given to racial and ethnic minorities early in biomarker development to ensure biomarkers are equally effective across all underserved and minority populations and are equally accessible by all.

Applicants hypothesized that the p-EMT signature may be correlated with sociodemographic disparities. To understand whether p-EMT was correlated with discrepancies in socioeconomic factors among head and neck squamous cell carcinoma (HNSCC) patients, Applicants utilized the p-EMT scores from TCGA to determine each racial cohort's median score. Non-white HNSCC patients were more likely to have a higher p-EMT expression among malignant cells (median: 0.24 [Interquartile Range (IQR): −0.65, 0.76] compared to White HNSCC (median: 0.02 [IQR: −0.59, 0.51]). Additionally, Black HNSCC patients with high p-EMT expression have almost 6.5 times the hazard of death, while White HNSCC patients with high p-EMT expression have almost 2 times the hazard of death. These findings in TCGA suggest differential p-EMT expression by at least one measure of socioeconomic status, namely race. However, TCGA is severely handicapped by the lack of racial diversity: There are only 47 Black American HNSCC patients with no socioeconomic status annotations and thus Applicants are unable to analyze OCSCC patients only (Table 4). Thus, to understand the potential interaction between p-EMT (as a surrogate of tumor biology/prognosis) and sociodemographic factors better, a more completely annotated, socioeconomically diverse cohort is essential (Table 6).

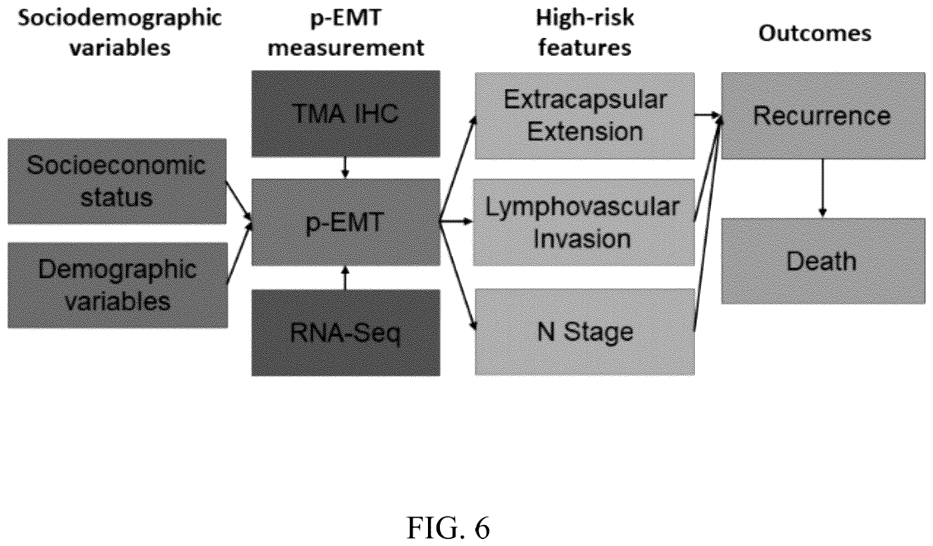

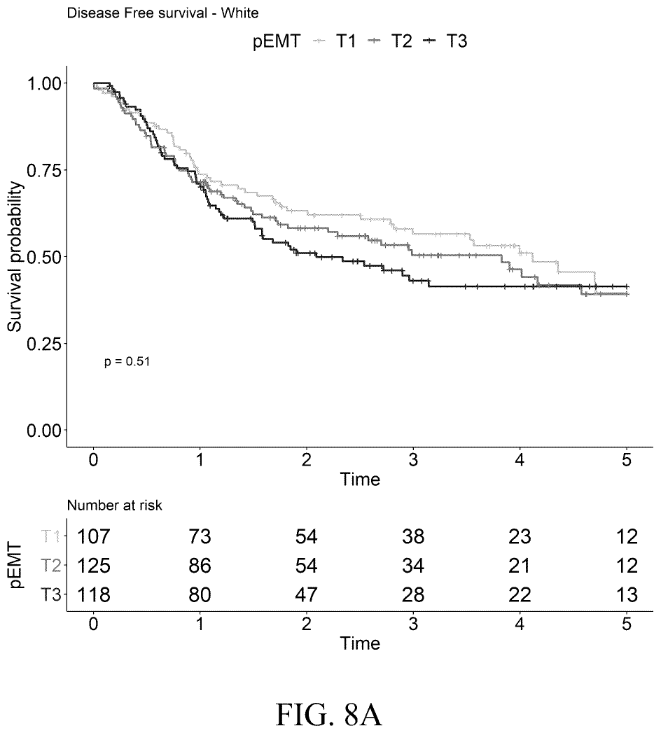

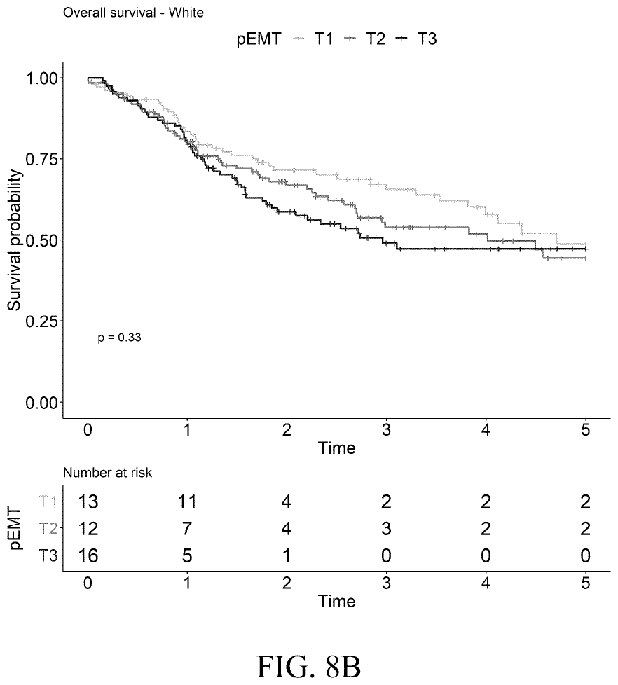

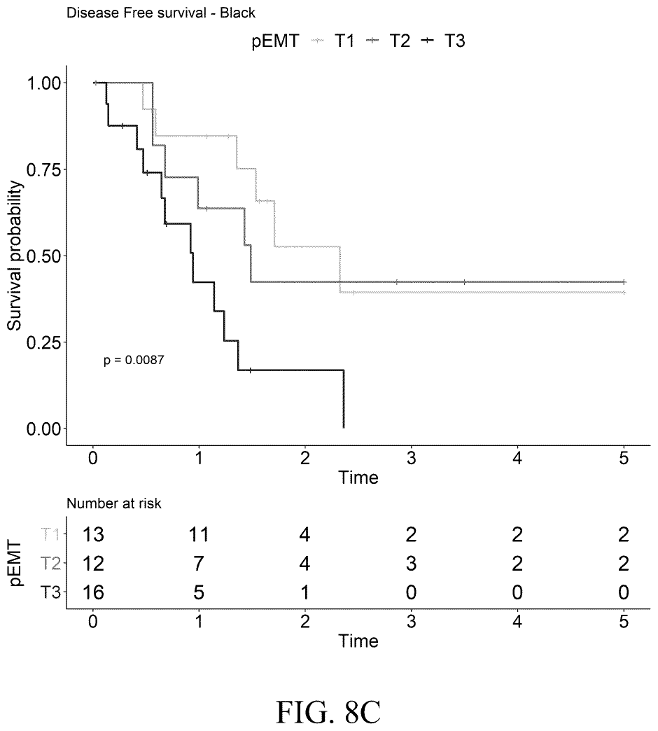

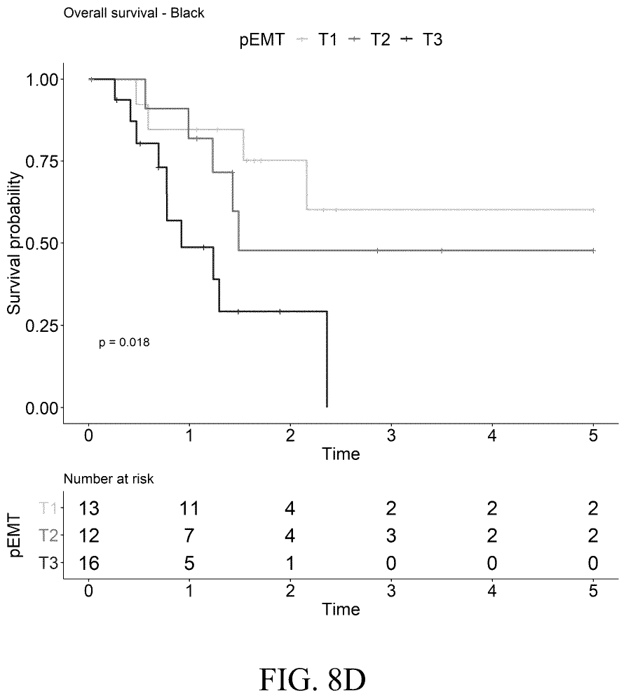

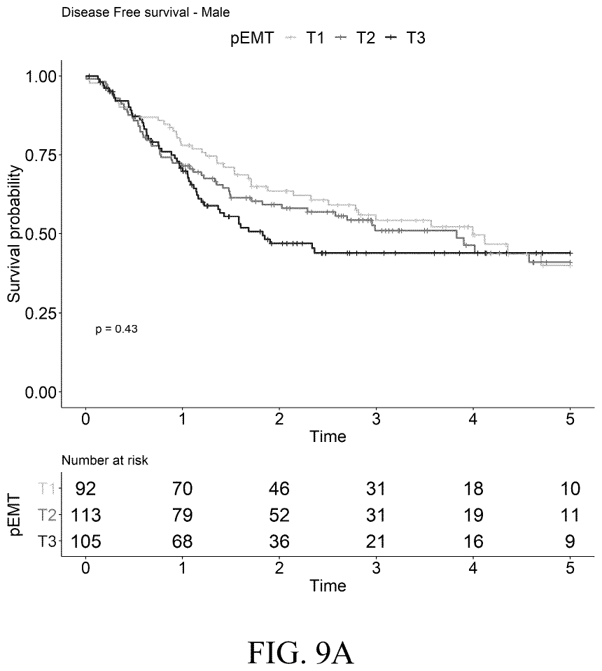

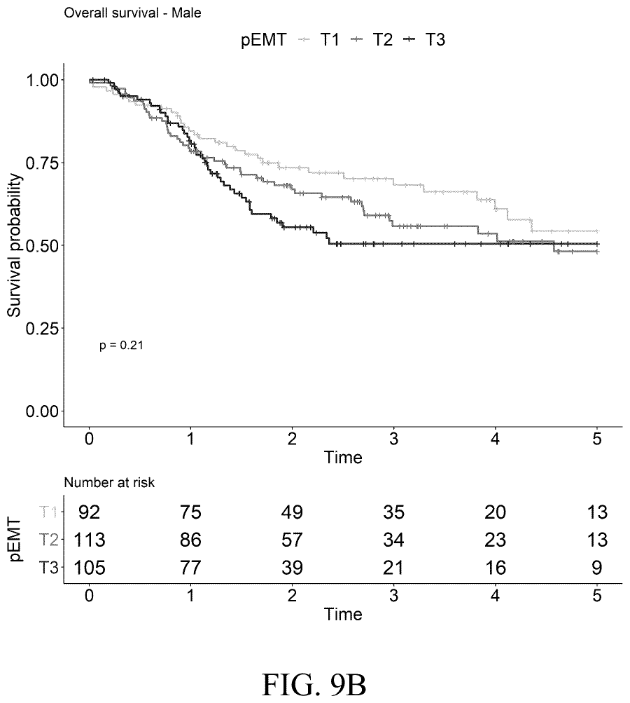

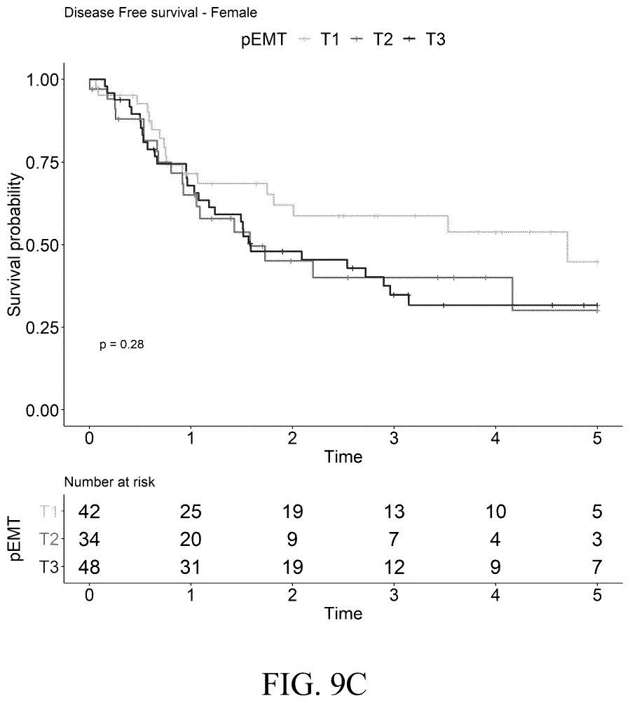

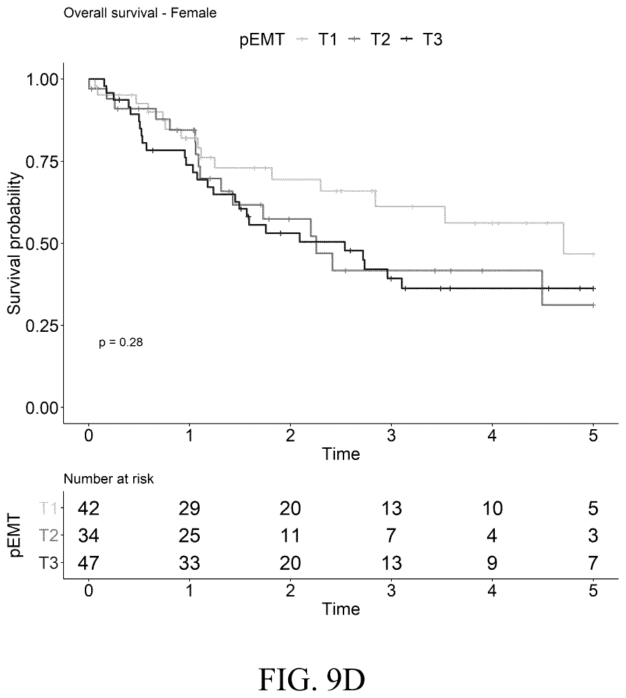

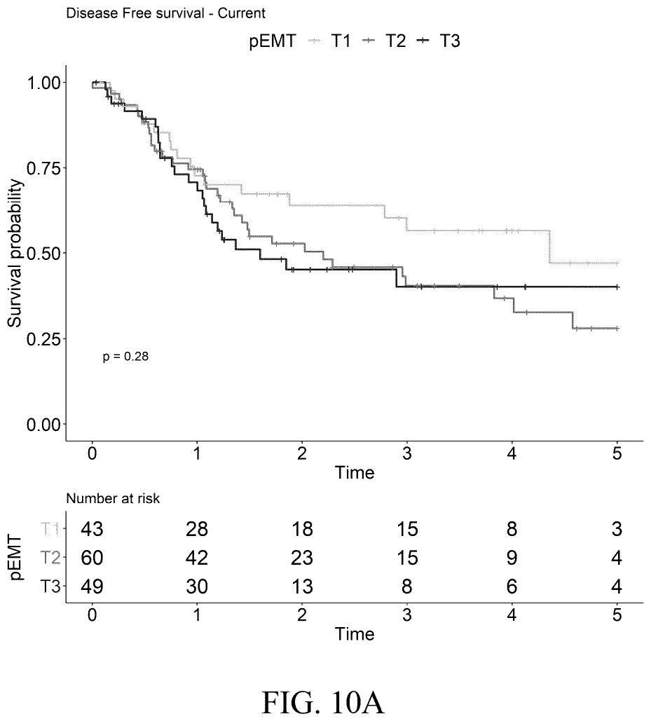

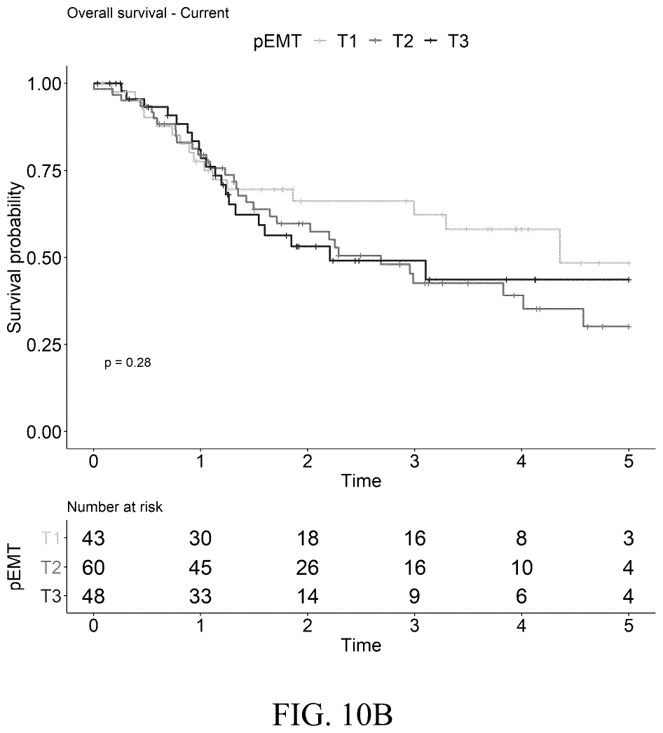

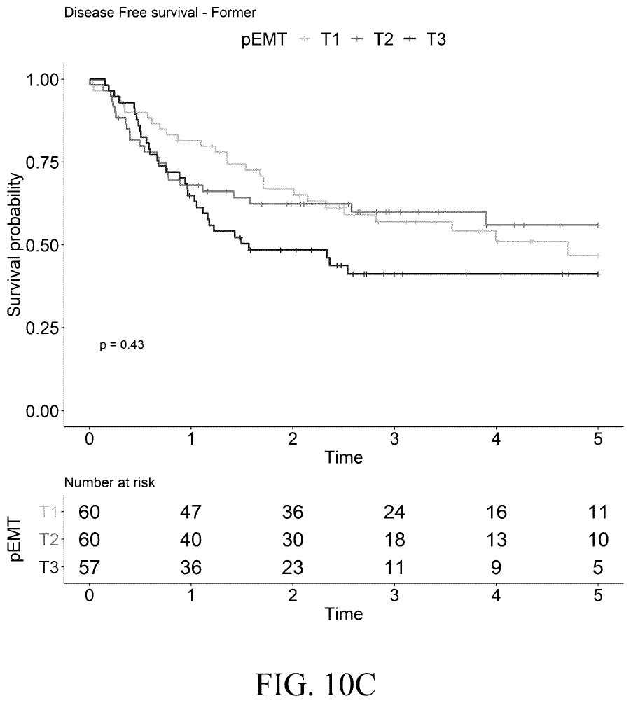

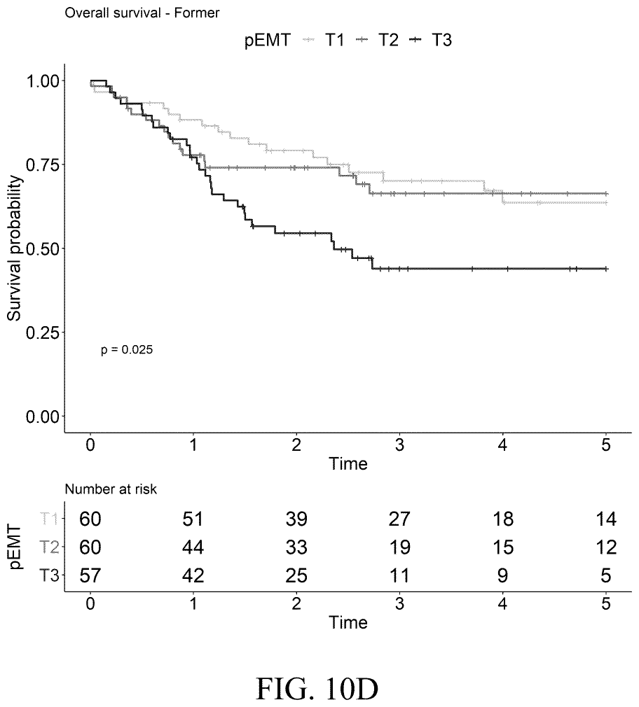

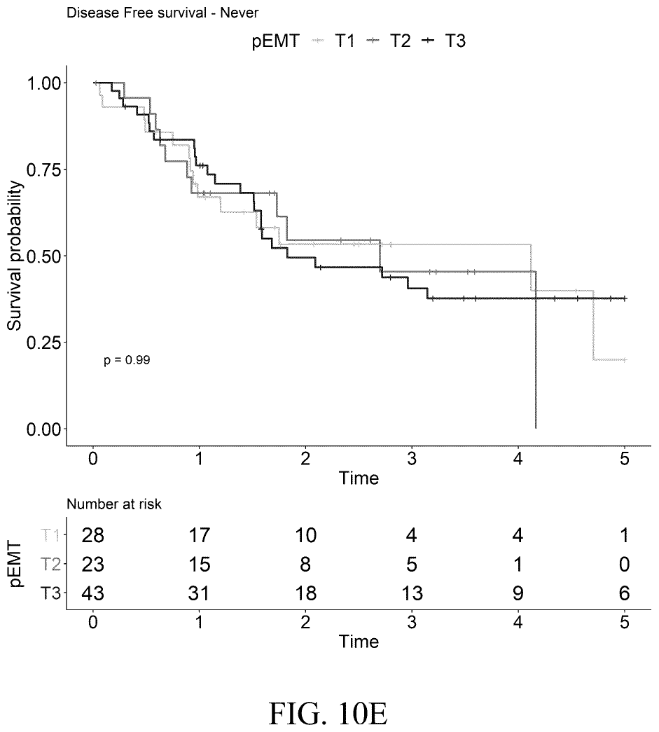

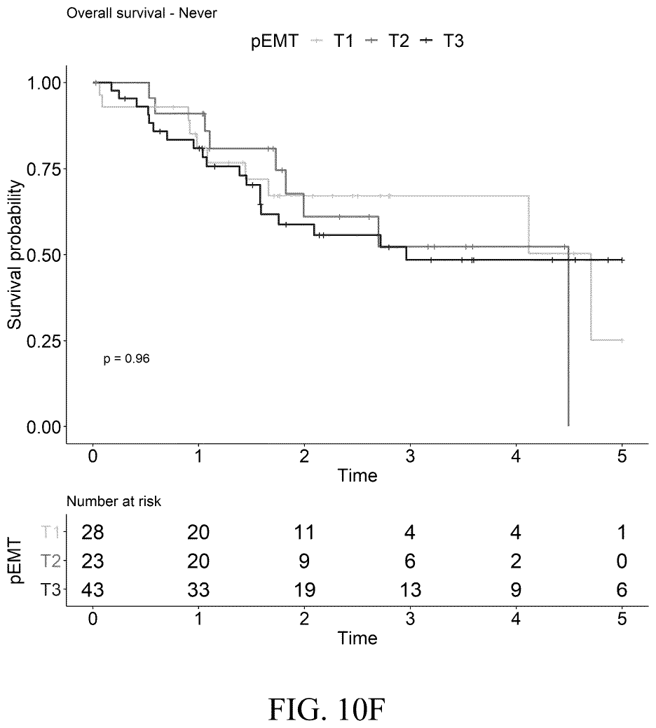

Preliminary results demonstrate that p-EMT significantly influences progression and outcome in OCSCC, which surpasses all other major risk factors to date, including smoking (FIG. 1). Further, p-EMT demonstrates differential expression and influence on survival by race using different models (Tables 5-10). Applicants hypothesized that p-EMT is a critical biologic prognosticator in OCSCC that may help clinicians bridge cancer health disparities by stratifying patients across diverse sociodemographic groups. The present invention uses a prospectively annotated, sociodemographically diverse cohort overall and within racial subgroups. The experimental design uses the cohort annotated for socioeconomic status and demographic variables (FIG. 6). The p-EMT score is measured with IHC and RNA-seq. High risk features and outcomes can be correlated to p-EMT score. Subjects determined to be at risk based on p-EMT score have different disease free and overall survival based on race (FIG. 8A-D), gender status (FIG. 9A-D), and smoking status (FIG. 10A-F). Thus, p-EMT risk can be calculated based on the specific demographic group to increase the prognostic value.

### Experimental Approach

Applicants have access to >400 OCSCC specimens in their institutional tumor bank, which have been prospectively collected and annotated from Siteman Cancer Center in St. Louis through the Tissue Acquisition Protocol. Siteman Cancer Center and Washington University School of Medicine is the only cancer center in Missouri that has a Comprehensive Cancer Center designation and a member of the National Comprehensive Cancer Network. Applicants can obtain tissue from 400 OCSCC specimens (200 Black and 200 White Americans) and perform bulk RNA-seq. Briefly, total RNA can be isolated using the RNeasy Mini Kit (Qiagen) with mRNA extracted from total RNA using a Dynal mRNA Direct kit. Applicants can confirm the quality of RNA by spectrophotometer and Bioanalyser (Agilent) to determine RNA integrity, followed by TruSeq mRNA library preparation (Illumina) and sequencing on the Illumina platform. Each condition will be sequenced to a depth of 30M reads. Based on these bulk RNA-seq signatures, Applicants can calculate an inferred malignant profile using the previously described logistic regression approach for all detected genes (Puram et al., 2017). Applicants can then calculate a p-EMT score using described p-EMT genes, similar to the preliminary results. Applicants can use both quartiles and a cutpoint analysis in R to split p-EMT scores into high and low expression groups. Applicants can correlate the p-EMT score with high-risk features in OCSCC, including stage, nodal metastasis, LVI, ECE, and PNI using chi-square analyses. Applicants can calculate adjusted prevalence ratios for each of the high-risk features with p-EMT signature via binomial regression to adjust for other potential confounders such as smoking or alcohol use. Applicants can also calculate 1) disease-specific survival, 2) recurrence and 3) overall survival within each stratum of p-EMT. Applicants expect OCSCC cases with a higher p-EMT score to have more high-risk features, and accordingly, higher rates of recurrence and mortality. Applicants can also do each of these analyses within racial groups, Black and White patient subgroups, to validate these findings not only across racial groups, but also within racial groups. Finally, Applicants can explore patients with no apparent nodal metastasis at the time of surgery (clinical NO) to determine if p-EMT is predictive of patients with nodal metastases by calculating the risk for occult nodal metastases and p-EMT signature with the binomial regression.

Next, to understand if IHC p-EMT markers can act as a surrogate for genomically-based p-EMT expression scores, Applicants can construct tissue microarrays (TMAs) (3 cores per sample). Applicants can stain using standard IHC techniques for the top ten p-EMT markers (e.g., LAMB3, LAMC2, PDPN, etc.) for which protocols are optimized. TMA specimens can be scored in a blinded semi-quantitative fashion (0-4+ intensity of staining and percentage of positive cells). Associations between the TMA scoring with the genomically-based p-EMT signature can be calculated using a Kruskal-Walis test in both the overall cohort and within racial subgroups.

Applicants can analyze 400 patients and expect 260 categorized as high p-EMT and 140 as low p-EMT. With this sample size, Applicants can have 80% power to detect a difference of 20% in clinical factors, which is reasonable given that 60% of high p-EMT and 40% of low p-EMT OCSCC cases have PNI. For the survival analysis, 156 events are required for adequate power (1-β>80%) to detect an HR of 1.6, which is realistic given recurrence-free survival in this population is 70% with >90% of events occurring by year 3. Additionally, if the TMA adequately approximates p-EMT, Applicants can acquire additional FFPE cases with longer follow-up times at low cost and be able to bolster the sample size. Applicants can also analyze bulk RNA-seq data to determine if specific changes in gene expression correlate with outcomes in the prospective cohort and validate these within racial subgroups. Applicants can also utilize the scRNA-seq algorithms (Puram et al., 2017) to deconvolve bulk data into cell type proportions (CIBERSORTx) and determine if certain cell types are enriched in patients with poor outcomes (e.g., CAFs, T_{reg}).

Applicants can abstract individual-level sociodemographic data such as insurance, gender, and race from the medical record and two neighborhood-level measures: 1) socioeconomic status and 2) rural-urban index. Applicants can calculate a geospatial score for neighborhood-level socioeconomic status using 21 variables from the 2010 US Census in six domains (education, employment and occupation, housing conditions, income and poverty, racial composition, and residential stability) based on the neighborhoods that contained at least one study participant. The index scores can be categorized into quartiles based on the distribution among census tracts and counties. For rural-urban index, Applicants can use Rural-Urban Commuting Area Codes (RUCAs) developed by the US Department of Agriculture's (USDA) to delineate rural-urban census tracts. This definition relies on a combination of area attributes, including population density (individuals/square mile), proximity to urban areas (defined by the census bureau as ≥50,000 persons/square mile), and daily commuting patterns. Applicants can classify RUCAs into four categories, urban, large rural, small rural, and isolated. If power becomes an issue, Applicants can collapse the rural variables. For consistency, Applicants can use RUCAs from 2010.

First, Applicants can use univariate statistics for each sociodemographic variable. For the significant variables, Applicants can use principal component analysis to determine each variable's contribution to the p-EMT score. Next, Applicants can utilize linear models to estimate the effect of neighborhood-level and individual-level variables on p-EMT signature. For the relationship between p-EMT and sociodemographic variables, Applicants can use multinomial logistic regression analysis to estimate odds ratios for the effect of neighborhood-level and individual-level variables on p-EMT signature. If any geospatial differences in the prevalence of HPV vaccination are identified in the unadjusted analyses, Applicants can consider the innate relationships of individuals within a neighborhood with a multilevel logistic regression or multilevel multinomial logistic regression, as appropriate. Applicants can also explore the relationship between p-EMT signature and survival among sociodemographic subgroups such as rural patients or Black American patients.

This analysis can demonstrate a higher degree of p-EMT among OCSCC minority and lower socioeconomic status patients, thus leading to poorer outcomes. With 80% power and an alpha of 0.05, Applicants can be powered to determine a moderate Cohen's D of 0.39. Moreover, if Applicants use quartiles for socioeconomic status, Applicants can detect a Cohen's D of 0.35. Applicants can also construct a TMA that oversamples minorities with additional FFPE cases. Additionally, Applicants can explore other metrics of neighborhood, including racial segregation, which is especially pertinent to St. Louis. Applicants can also analyze bulk RNA-seq data to determine if specific changes in gene expression correlate with race or other sociodemodraphics.

### DISCUSSION

The prognostic value of p-EMT is highly promising with analyses from TCGA, suggesting it is a more reliable predictor of outcome than smoking, especially among underrepresented subgroups. However, markers that are developed with the idea of sociodemographic diversity and within demographic groups at the outset have not been pursued. The findings presented herein could establish a novel paradigm in which patients are tested for p-EMT, which dictates intensification or de-intensification of their treatment regimen, along with more informed, shared treatment decision-making. Investigations of p-EMT in OCSCC may represent a rich source of new drug targets. Applicants can develop a narrowed immunohistochemical panel for assessing p-EMT that will allow its broader use as a biomarker and will prevent widening head and neck disparities. These studies could make p-EMT accessible to any pathologist capable of performing IHC rather than requiring expensive, logistically challenging genomic analyses, which will ensure p-EMT only be available to those who can afford it. While disparities across the cancer continuum have been well-documented, the interaction with molecular markers has not. As precision medicine becomes a reality, how social “omics” influence prognostication and interact with tumor genomics becomes more critical than ever. Prognostic breast cancer molecular markers, for example, interact with stressors from the environment, suggesting both social and molecular determinants affect prognostication. The p-EMT signature can be used as molecular markers for social determinants of health as patient-specific medicine advances health disparities will widen.

Despite significant gains in the molecular understanding of OCSCC over the past several decades, little progress has been made in utilizing this information to improve patient outcomes or stratify patients based on their underlying disease biology. Applicants' results indicate that the p-EMT state among malignant cells is highly predictive of poor overall survival, disease progression (nodal metastasis), and adverse histopathologic features (LVI, ECE, grade). Importantly, p-EMT is more strongly associated with survival than T or N stage, age, race, or smoking status. The data indicates that p-EMT status may predict occult nodal disease, undetectable via standard clinical methods. Thus, this p-EMT program holds great promise as a diagnostic biomarker for risk stratification. The external validity of p-EMT as a marker in OCSCC is limited by confirmation of its prognostic value in an orthogonal dataset. Applicants can validate p-EMT as a predictor of adverse histopathologic features and patient outcomes using a prospectively collected and annotated diverse cohort of OCSCC patients and within racial subgroups. Applicants can determine which genes in the p-EMT program may be used in IHC analyses as a surrogate for a genomically-derived p-EMT score. Applicants can determine how the p-EMT signature differs within sociodemographic factors, specifically race, gender, and socioeconomic status. Additionally, Applicants can analyze the interaction between p-EMT signature and sociodemographic variables with recurrence and survival.

### Example 3—p-EMT Score and HNSCC

A High p-EMT Score was Associated with Poor Oncologic Features in HNSCC

In this cohort, there were 309 oral cavity cancer patients, 125 laryngeal cancer patients, and 47 HPV(+) oropharyngeal cancers (Table 4) diagnosed from 2011 to 2015. Oral cavity patients with nodal metastasis or perineural invasion (PNI) had higher primary tumor p-EMT scores than patients without nodal metastasis [mean: 0.20 (standard deviation (std): 0.86) v. mean: −0.03 (std: 0.92)] or PNI [mean: 0.25 (std: 0.79) v. mean: −0.09 (std: 0.95)]. The tumor subtype was also associated with p-EMT score. Tumors with mesenchymal and basal subtypes have higher p-EMT scores in laryngeal and oral cavity cancers. When exploring tertile categories of p-EMT, the trends continued to hold (Table 6).

Even when Adjusting for Poor Clinical Features, High p-EMT was Associated with Poor Overall and Disease-Free Survival in Oral Cavity and Laryngeal Cancers.

Patients with high p-EMT had worse survival in both laryngeal and oral cavity cancer (FIG. 7A, B). The five-year overall survival for oral cavity cancer patients with high p-EMT was 41.1% [95% Confidence Interval (CI): 33.3-50.8%] and low p-EMT was 51.4% (95% CI: 38.9-68.0%). Similarly, the five-year survival was significantly lower for laryngeal cancer patients with high p-EMT [OS: 25.2% (95% CI: 11.8%-53.8%)] than patients with low p-EMT [OS: 61.7% (95% CI: 51.3%-74.3)]. When using tertiles for p-EMT score, there appeared to be a dose-response for laryngeal cancer where higher p-EMT was associated with poorer survival. In oral cavity cancer, patients in the lowest tertile had the best survival, with no difference in survival between the two lowest tertiles. Applicants found that p-EMT did not appear to be associated with survival in HPV-positive oropharyngeal cancer.

The adjusted Cox Regression (Table 11) with imputed data demonstrated similar results to the univariate analysis. Laryngeal cancer patients with high p-EMT have a greater risk of death [HR: 4.26 (95% CI 1.90, 9.55)] and recurrence/death [HR: 3.21 (95% CI: 1.58, 6.49)]. Although attenuated, oral cavity patients with high p-EMT have 1.27 (95% CI: 0.87, 1.86) times the risk of death and 1.45 (95% CI: 0.95, 2.20) times the risk of recurrence or death compared with patients with low p-EMT. Additionally, when Applicants did not adjust for LVI and PNI, the association did not change (Table 13 and 14) for overall survival. However, adjusting for LVI and PNI modestly diminished the association between p-EMT and DFS.

Black Americans with High p-EMT Scores had Worse Survival than White Americans with High p-EMT Scores.

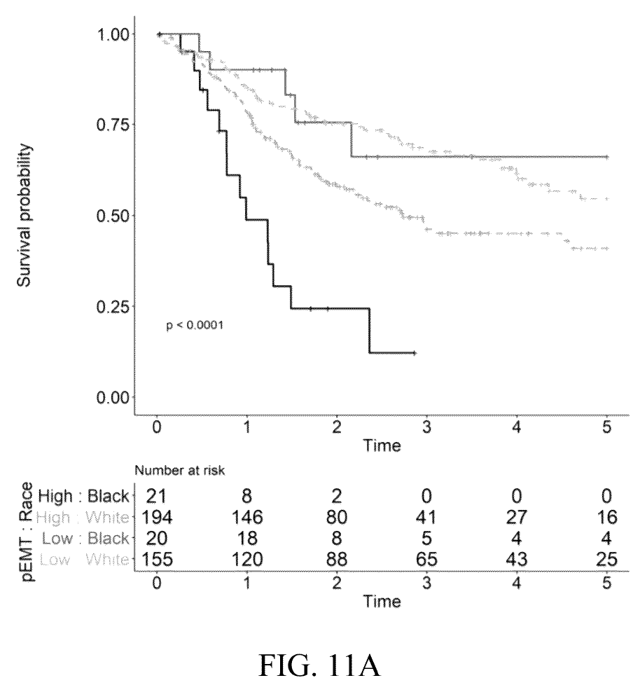

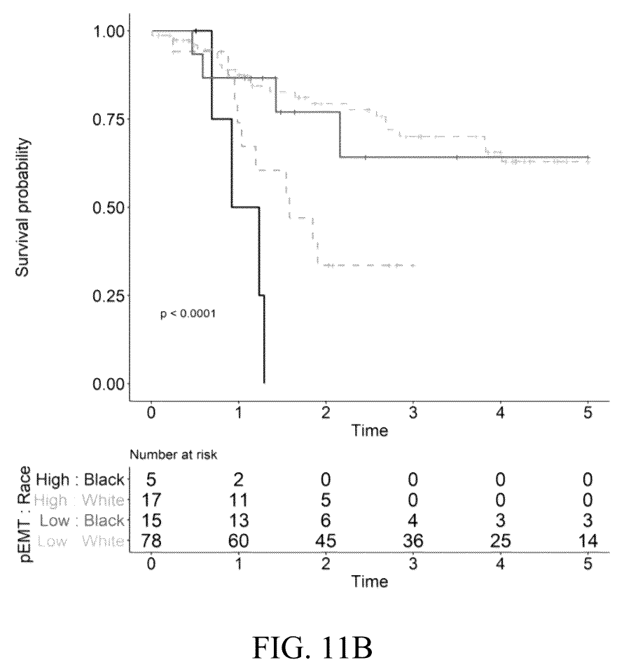

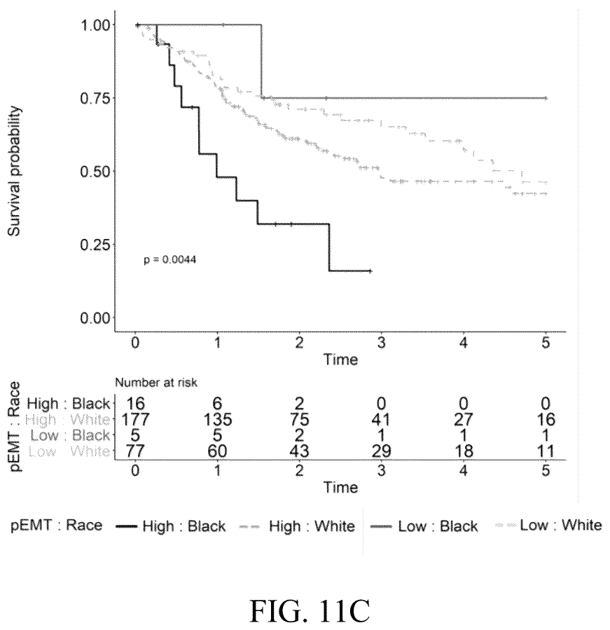

Applicants next investigated the potential effect modification of race with p-EMT. p-EMT is strongly associated with survival among White and Black patients. However, Black patients with a high p-EMT score have worse five-year survival than White patients, especially among those with oral cavity cancer (FIG. 11A, B, C). After adjusting for smoking, T stage, N stage, PNI, LVI and site (Table 3), Black patients with low p-EMT score had similar survival to White patients with low p-EMT score [HR: 0.96 (95% CI: 0.38, 2.43)]. However, compared to White patients with low p-EMT score, Black patients with high p-EMT score have a higher risk of death [4.14 (95% CI: 2.17, 7.91)] than White patients with high p-EMT score [1.68 (95% CI: 1.12, 2.52)]. However, this interaction is not significant (p=0.11). Results with DFS are similar to OS (Table 12), with the interaction between race and p-EMT approaching significance (p=0.060).

### DISCUSSION

There are no objective markers to predict outcomes in HPV-negative HNSCC. Thus, clinicians rely on traditional tumor stage and histopathologic markers such as lymphovascular invasion, extranodal extension, and perineural invasion. These histopathologic markers are difficult to standardize because of problems inherent in the visual analysis and subjective definition. Applicants observed p-EMT was more strongly associated with overall and disease-free survival than smoking, tumor/nodal stage, or other well-established, adverse histopathologic features. Notably, there appears to be an interaction between race and p-EMT, suggesting the possibility of a genomic basis for racial and socioeconomic health disparities in HNSCC. These data highlight a potential new marker for treatment stratification a priori in HPV-negative HNSCC.

The classical model of EMT is one in which malignant cells shed their epithelial identity and adopt a mesenchymal phenotype. EMT is characterized by loss of cell polarity, motility, and ability to remodel the extracellular matrix, and ultimately increased invasive potential (Lambert A W, Pattabiraman D R, Weinberg R A. Emerging Biological Principles of Metastasis. Cell. 2017; 168(4):670-91). An increasing body of recent scientific work has revealed a more nuanced picture. Subpopulations of malignant cells simultaneously harbor epithelial and mesenchymal features. These subpopulations are termed p-EMT (otherwise referred to as “hybrid EMT” or “EMT plasticity”) (Pastushenko I, Blanpain C. EMT Transition States during Tumor Progression and Metastasis. Trends Cell Biol. 2019; 29(3):212-26). p-EMT cells are better able to invade locally (Puram, et al., 2017), collectively migrate (Campbell K, et al., Collective cell migration and metastases induced by an epithelial-to-mesenchymal transition in Drosophila intestinal tumors. Nature communications. 2019; 10(1):2311), survive in the circulation as oligocellular clusters (Aceto N, et al., Circulating tumor cell clusters are oligoclonal precursors of breast cancer metastasis. Cell. 2014; 158(5):1110-22), and seed metastatic colonies (Pastushenko I, et al., Identification of the tumour transition states occurring during EMT. Nature. 2018; 556(7702):463-8).

p-EMT is significantly associated with the presence of lymph node metastases, multiple lymph node metastases, advanced nodal stage, higher grade, extracapsular extension (ECE), and lymphovascular invasion (LVI) (Parikh, et al., 2019). This has been supported by in vitro studies where two HNSCC cell lines separated in p-EMT high and low subpopulations based on the expression of surface markers as identified in single-cell analysis. Applicants found that the p-EMT population was significantly more invasive than control (unsorted) cells, while the non-p-EMT cells were less invasive. Applicants have previously determined that among patients with clinically node-negative disease (cN0), high p-EMT was strongly predictive of occult nodal disease ultimately found on surgical pathology (Puram, et al., 2017; and Schinke H, et al., Digital scoring of EpCAM and slug expression as prognostic markers in head and neck squamous cell carcinomas. Mol Oncol. 2021; 15(4):1040-53). Together, these results uncover a striking association between the p-EMT state and adverse histopathologic features, including metastasis in HNSCC.

To date, Applicants have the most robust epidemiologic and clinical analysis linking p-EMT with HNSCC survival. However, a few previous studies have also demonstrated the association between p-EMT and overall survival in head and neck cancers (Wangmo, et al., 2020; Kisoda et al., 2020; and Schinke, et al., 2021). Kisoda et al., also using TCGA, found SERPINE1, ITGA5, TGFBI, P4HA2, CDH13, LAMC2, and MT1X significantly associated with survival in HNSCC patients. However, this study included all HNSCC sites and did not perform a multivariable analysis. Another study with OCSCC patients from a hospital in Thailand used TMAs and IHC to define EMT as no, partial and complete (Wangmo, et al., 2020). Wangmo et al. found a dose-response relationship in which complete EMT has the worst survival followed by partial EMT, as measured by Vimentin and E-cadherin on IHC. However, this analysis did not use single-cell RNA, limiting the generalizability of this current study.

There has been a stronger push for more diverse studies with a definitive study of race and the variations of social determinants of health in genomic research (Sirugo G, Williams S M, Tishkoff S A. The Missing Diversity in Human Genetic Studies. Cell. 2019; 177(1):26-31). The findings herein in TCGA suggest p-EMT has an exaggerated association with poor survival among Black Americans. However, differences by race do not necessarily mean there are biological differences in race. Race is a social and power construct, and self-reported race is a poor approximation of human genetic variation and ancestry (Yudell M, Roberts D, DeSalle R, Tishkoff S. SCIENCE AND SOCIETY. Taking race out of human genetics. Science. 2016; 351(6273):564-5). Given race's correlation with many social and societal aspects, the results here potentially represent effects of structural racism and the embodiment of external stressors (i.e., allostatic load). Black Americans have higher rates of comorbidities (Mazul A L, Naik A N, Zhan K Y, Stepan K O, Old M O, Kang S Y, Nakken E R, Puram S V. Gender and race interact to influence survival disparities in head and neck cancer. Oral Oncol. 2021; 112:105093), less likely to receive adherent treatment (Nocon C C, Ajmani G S, Bhayani M K. A contemporary analysis of racial disparities in recommended and received treatment for head and neck cancer. Cancer. 2020; 126(2):381-9), and accelerated epigenetic aging (Brody G H, Yu T, Beach S R. Resilience to adversity and the early origins of disease. Dev Psychopathol. 2016; 28(4pt2):1347-65). All of these, plus many other yet unexplored factors, can contribute to the race-p-EMT interaction seen in these data. Additional studies should determine if this association is due to ancestry or social factors associated with race and racism.

This study has a few limitations. First, TCGA did not collect socioeconomic data beyond race. Thus, Applicants are unable to estimate the effect of race or socioeconomic status. This lack of diversity did not allow us to stratify results beyond Black and White Americans. Second, many clinical variables in TCGA are missing. While Applicants have imputed LVI and PNI, treatment variables still have high rates of missing and could not be assessed. However, Applicants anticipate that all patients were adherent to treatment guidelines. Third, the generalizability of TCGA may be limited, especially given the lack of racial diversity and that most patients are higher stage at diagnosis. However, the strengths outweigh the benefits of TCGA. Together, the findings identify an essential signature that can be readily applied to HNSCC patient samples and discern important underlying biological states while more broadly emphasizing the potential translational impact of single-cell data across oncology and medicine.

### Methods

Data and specimen collection. Bulk RNA-sequencing and clinical data of HNSCC tumors were obtained from The Cancer Genome Atlas (TCGA) database (portal.gdc.cancer.gov/(accessed on 1 Mar. 2020)). Applicants included oral cavity, laryngeal and HPV-positive oropharyngeal squamous cell carcinoma. HPV-positive oropharyngeal cancers have substantially better survival than HPV-negative cancers. Applicants excluded HPV-negative oropharyngeal cancer due to the small sample size (N=11).

Exposure assessment. To investigate how intra-tumoral heterogeneity is associated with patient outcomes (Puram, et al. 2015; and Baba S, et al., Global DNA hypomethylation suppresses squamous carcinogenesis in the tongue and esophagus. Cancer Sci. 2009; 100(7):1186-91), HNSCC expression heterogeneity was characterized at single-cell resolution. Applicants used scRNA-seq (SMART-seq2) (Picelli S, et al., Full-length RNA-seq from single cells using Smart-seq2. Nat Protoc. 2014; 9(1):171-81) to profile ˜6,000 cells from primary HNSCC and matched lymph node (LN) metastases (Puram et al., 2017). Applicants separated malignant and non-malignant cells using 1) expression of epithelial markers (e.g., cytokeratins) and 2) presence or absence of copy number variations (CNVs) (Patel A P, et al., Single-cell RNA-seq highlights intratumoral heterogeneity in primary glioblastoma. Science. 2014; 344(6190):1396-401; Tirosh I, et al., Dissecting the multicellular ecosystem of metastatic melanoma by single-cell RNA-seq. Science. 2016; 352(6282):189-96; Tirosh I, et al., Single-cell RNA-seq supports a developmental hierarchy in human oligodendroglioma. Nature. 2016; 539(7628):309-13; and Venteicher A S, et al., Decoupling genetics, lineages, and microenvironment in IDH-mutant gliomas by single-cell RNA-seq. Science. 2017; 355(6332)). Applicants then broadly clustered malignant and non-malignant cells using multidimensional, t-distributed stochastic neighbor embedding analysis (t-SNE). These analyses showed that while non-malignant cells clustered across tumors, malignant cells clustered almost exclusively by their tumor of origin. For malignant cells, Applicants clustered profiles from individual tumors and uncovered subpopulations related to the cell cycle, epithelial differentiation, stress, hypoxia, and an EMT-like program, which has some features of EMT (e.g., VIM, ITGA5), yet retains expression of epithelial markers and expresses Snail2 but not other classical EMT TFs (Diepenbruck M, Christofori G. Epithelial-mesenchymal transition (EMT) and metastasis: yes, no, maybe? Curr Opin Cell Biol. 2016; 43:7-13; Nieto M A, et al., Emt: 2016. Cell. 2016; 166(1):21-45; and Revenu C, Gilmour D. EMT 2.0: shaping epithelia through collective migration. Curr Opin Genet Dev. 2009; 19(4):338-42). Applicants, therefore, considered this latter program a partial EMT (p-EMT) program. When this analysis was applied to all tumors, Applicants observed that these programs were remarkably consistent (Puram et al. 2017), suggesting sub-populations were robust. Applicants analyzed the large bulk RNA-seq repository from the Cancer Genome Atlas (TCGA) and deconvolved these data using the single-cell profiles to correlate disease progression with p-EMT.

Applicants also obtained clinical and demographic data from TCGA. For clinical variables Applicants used AJCC 7^{th} edition Tumor, Nodal and Metastasis stage, lymphovascular invasion, and perineural invasion. Race and ethnicity were self-reported. Race and ethnicity were grouped together. Applicants classified patients as Hispanic if reported Hispanic regardless of race. Applicants categorized race/ethnicity into non-Hispanic White and non-White, including Black, Asian, Hispanic and other. Applicants excluded patients missing race. Smoking was grouped into current, former, and never.

Outcome assessment. The primary outcome was overall survival. Applicants also studied disease-free survival (DFS) as a secondary outcome. Applicants used the time from diagnosis to death for overall survival or the last known contact available from the TCGA. DFS was defined as the interval from diagnosis to the date of progression, date of recurrence, death, or date of last known contact if the patient was alive and has not recurred.

Statistical Analysis. The prognosis of each group of patients was examined by Kaplan-Meier survival, and log-rank tests compared the survival outcomes. Kaplan-Meier plots are presented for p-EMT. Using the Cox proportional-hazards model, hazard ratios (HRs) and corresponding 95% confidence intervals (CIs) for risk of disease progression and mortality associated with p-EMT. Applicants used complete case analysis with the Multivariable Cox model adjusted for tumor stage, race, smoking status, and age.

In addition to analyzing survival by tertiles, Applicants also performed Cut point analysis using the R package survminer (0.4.2). Due to the small sample size, Applicants could not do a cutpoint analysis in HPV+ oropharyngeal cancer.

Since 28.3% of the cases were missing LVI and 26.0% missing PNI, Applicants also multiply imputed the missing data for these variables and smoking, N stage, and T stage. Applicants multiply imputed using additive regression, bootstrapping, and predictive mean matching with areglmpute function in the hmisc package (Alzola C, Harrell F. An introduction to S and the Hmisc and design libraries. Departement of biostatistics, Vanderbilt University biostat me vanderbuilt edu/RS/sintro pdf (accessed 2013/05). 2006). Applicants pooled 100 Cox proportional regression imputations with Rubin's rules.

Additionally, Applicants explored a possible interaction with self-reported race. Applicants fully stratified p-EMT with race but excluded non-White or non-Black race. To maintain sample size, Applicants combined all sites but maintained the high/low p-EMT categorization by site.

All statistical analyses were performed using R version 3.6.

TABLE 4
Descriptive statistics for pEMT for oral cavity, larynx and HPV(+) oropharynx cancer.

| | Oral Cavity | | | | Larynx | | | | HPV(+) Oropharynx | | | |
|---|---|---|---|---|---|---|---|---|---|---|---|---|
| | N | Mean (SD) | | p* | N | Mean (SD) | | p* | N | Mean (SD) | | p* |
| Gender | | | | | | | | | | | | |
| Female | 101 | 0.10 | (0.90) | 0.646 | 23 | −0.25 | (1.08) | 0.092 | 5 | 0.39 | (0.81) | 0.015 |
| Male | 208 | 0.05 | (0.90) | | 102 | 0.09 | (0.82) | | 42 | −0.52 | (0.75) | |
| Race | | | | | | | | | | | | |
| Non-White | 48 | 0.17 | (0.91) | 0.399 | 26 | −0.10 | (1.11) | 0.451 | 3 | 0.14 | (0.26) | 0.219 |
| White | 255 | 0.05 | (0.87) | | 95 | 0.05 | (0.82) | | 44 | −0.46 | (0.81) | |
| Missing | 6 | −0.07 | (1.8) | | 4 | 0.41 | (0.67) | | — | — | | |
| Smoking | | | | | | | | | | | | |
| Current | 93 | 0.02 | (0.88) | 0.624 | 59 | 0.04 | (0.78) | 0.093 | 9 | −0.37 | (0.68) | 0.918 |
| Former | 122 | 0.03 | (0.90) | | 55 | −0.05 | (0.94) | | 20 | −0.40 | (0.79) | |
| Never | 86 | 0.14 | (0.95) | | 8 | 0.67 | (0.82) | | 17 | −0.49 | (0.93) | |
| Missing | 8 | 0.24 | (0.72) | | 3 | −0.52 | (1.35) | | 1 | −0.09 | (—) | |
| N Stage | | | | | | | | | | | | |
| N | 164 | −0.03 | (0.92) | 0.032 | 58 | −0.04 | (0.85) | 0.532 | 9 | −0.33 | (0.96) | 0.719 |
| N+ | 133 | 0.20 | (0.86) | | 60 | 0.06 | (0.89) | | 38 | −0.44 | (0.77) | |
| Missing | 12 | 0.07 | (1.03) | | 7 | 0.35 | (1.09) | | 0 | — | | |
| T Stage | | | | | | | | | | | | |
| T1-T2 | 114 | 0.12 | (0.87) | 0.397 | 23 | −0.04 | (0.95) | 0.661 | 31 | −0.51 | (0.86) | 0.359 |
| T3-T4 | 187 | 0.03 | (0.92) | | 98 | 0.05 | (0.87) | | 15 | −0.28 | (0.67) | |
| Missing | 8 | | | | 4 | −0.07 | (0.98) | | 1 | 0.27 | (—) | |
| LVI | | | | | | | | | | | | |
| No | 161 | 0.04 | (0.90) | 0.445 | 44 | −0.13 | (0.84) | 0.231 | 6 | 0.06 | (0.87) | 0.065 |
| Yes | 69 | 0.14 | (0.86) | | 37 | 0.08 | (0.75) | | 10 | −0.84 | (0.87) | |
| Missing | 79 | 0.04 | (0.95) | | 44 | 0.15 | (1.00) | | 31 | −0.38 | (0.73) | |
| PNI | | | | | | | | | | | | |
| No | 109 | −0.09 | (0.95) | 0.003 | 49 | −0.16 | (0.78) | 0.119 | 16 | −0.34 | (0.77) | NA |
| Yes | 134 | 0.25 | (0.79) | | 29 | 0.13 | (0.80) | | 1 | −1.63 | (—) | |
| Missing | 66 | −0.06 | (0.98) | | 47 | 0.16 | (0.99) | | 30 | −0.42 | (0.81) | |
| Subtype | | | | | | | | | | | | |
| Atypical | 35 | −0.59 | (0.81) | <0.001 | 48 | −0.13 | (0.78) | 0.02 | 45 | −0.45 | (0.81) | 0.252 |
| Basal | 128 | 0.13 | (0.80) | | 12 | 0.18 | (1.33) | | — | — | | |
| Classical | 43 | −0.10 | (0.91) | | 38 | −0.12 | (0.86) | | 2 | 0.22 | (0.29) | |

| | | | | | | | | |
|---|---|---|---|---|---|---|---|---|
| Mesenchymal | 103 | 0.27 | (0.94) | 27 | 0.46 | (0.70) | — | — |

LVI: Lymphovascular Invasion;
PNI: Perineural invasion
*t-test p-value for pEMT score not including the missing

TABLE 5
Adjusted hazard ratio for overall survival and disease-free survival for larynx and oral cavity cancer in the imputed dataset.

| | Larynx Cancer | | | | Oral Cavity Cancer | | | | |
|---|---|---|---|---|---|---|---|---|---|
| | Overall Survival | | Disease free survival | | | Overall Survival | | Disease free survival | |
| | HR (95% CI) | p | HR (95% CI) | p | | HR (95% CI) | p | HR (95% CI) | p |
| pEMT | | | | | pEMT | | | | |
| Low | 1 | | 1 | | Low | 1 | | 1 | |
| High (>0.6169) | 2.50 (1.00, 6.25) | 0.055 | 2.61 (1.26, 5.41) | 0.010 | High (>−0.3877) | 1.49 (0.98, 2.26) | 0.055 | 1.39 (0.96, 2.02) | 0.084 |
| Smoking | | | | | Smoking | | | | |
| Current | 1 | | 1 | | Current | 1 | | 1 | |
| Former | 0.42 (0.20, 0.90) | 0.028 | 0.86 (0.47, 1.57) | 0.626 | Former | 0.70 (0.45, 1.09) | 0.028 | 0.75 (0.50, 1.14) | 0.178 |
| Never | 2.73 (0.93, 7.98) | 0.067 | 2.06 (0.72, 5.88) | 0.176 | Never | 0.73 (0.45, 1.17) | 0.067 | 0.88 (0.56, 1.37) | 0.572 |
| T stage | | | | | T stage | | | | |
| T1-T2 | 1 | | 1 | | T1-T2 | 1 | | 1 | |
| T3-T4 | 1.39 (0.49, 3.92) | 0.538 | 1.29 (0.57, 2.94) | 0.541 | T3-T4 | 1.18 (0.79, 1.76) | 0.538 | 1.26 (0.87, 1.83) | 0.221 |
| N Stage | | | | | N Stage | | | | |
| N0 | 1 | | 1 | | N0 | 1 | | 1 | |
| N+ | 1.04 (0.41, 2.65) | 0.935 | 1.23 (0.66, 2.31) | 0.516 | N+ | 1.13 (0.76, 1.68) | 0.935 | 1.15 (0.8, 1.65) | 0.452 |
| Race | | | | | Race | | | | |
| Non-White | 1 | | 1 | | Non-White | 1 | | 1 | |
| White | 0.42 (0.19, 0.92) | 0.031 | 0.66 (0.33, 1.32) | 0.244 | White | 0.71 (0.44, 1.17) | 0.031 | 0.68 (0.44, 1.05) | 0.081 |
| PNI | | | | | PNI | | | | |
| No | 1 | | 1 | | No | 1 | | 1 | |
| Yes | 2.66 (0.77, 9.19) | 0.150 | 1.74 (0.65, 4.66) | 0.286 | Yes | 1.58 (0.95, 2.62) | 0.622 | 1.20 (0.8, 1.82) | 0.384 |
| LVI | | | | | LVI | | | | |
| No | 1 | | 1 | | No | 1 | | 1 | |
| Yes | 1.26 (0.50, 3.17) | 0.622 | 0.86 (0.34, 2.22) | 0.763 | Yes | 1.47 (0.92, 2.35) | 0.150 | 1.51 (1.02, 2.22) | 0.042 |
| Age | 1.01 (0.97, 1.05) | 0.665 | 1.00 (0.97, 1.03) | 0.962 | Age | 1.03 (1.01, 1.04) | 0.665 | 1.03 (1.01, 1.04) | 0.001 |

TABLE 6
Descriptive statistics by tertile and site.

| | Larynx | | | | Oral Cavity | |
|---|---|---|---|---|---|---|
| | T1 | T2 | T3 | | T1 | T2 |
| | (<−0.268) | (−0.268-0.328) | (>0.328) | | (<−0.305) | (−0.305-0.411) |
| | 41 | 42 | 42 | | 103 | 103 |
| | N (%) | N (%) | N (%) | p | N (%) | N (%) |
| Gender | | | | | | |
| Female | 4 (9.8) | 12 (28.6) | 7 (16.7) | 0.081 | 33 (32.0) | 32 (31.1) |
| Male | 37 (90.2) | 30 (71.4) | 35 (83.3) | | 70 (68.0) | 71 (68.9) |
| Race | | | | | | |
| Non-White | 5 (12.8) | 10 (23.8) | 11 (27.5) | 0.256 | 12 (11.8) | 15 (15.0) |
| White | 34 (87.2) | 32 (76.2) | 29 (72.5) | | 90 (88.2) | 85 (85.0) |
| Smoking | | | | | | |
| Current | 20 (48.8) | 20 (50.0) | 19 (46.3) | 0.478 | 39 (39.0) | 27 (26.7) |
| Former | 20 (48.8) | 18 (45.0) | 17 (41.5) | | 38 (38.0) | 46 (45.5) |
| Never | 1 (2.4) | 2 (5.0) | 5 (12.2) | | 23 (23.0) | 28 (27.7) |
| N Stage | | | | | | |
| N | 16 (41.0) | 24 (60.0) | 18 (46.2) | 0.217 | 56 (55.4) | 61 (62.2) |
| N+ | 23 (59.0) | 16 (40.0) | 21 (53.8) | | 45 (44.6) | 37 (37.8) |
| T Stage | | | | | | |
| T1-T2 | 10 (24.4) | 8 (20.0) | 5 (12.5) | 0.387 | 41 (40.2) | 34 (34.0) |
| T3-T4 | 31 (75.6) | 32 (80.0) | 35 (87.5) | | 61 (59.8) | 66 (66.0) |
| LVI | | | | | | |
| No | 15 (53.6) | 16 (57.1) | 13 (52.0) | 0.928 | 53 (70.7) | 55 (71.4) |
| Yes | 13 (46.4) | 12 (42.9) | 12 (48.0) | | 22 (29.3) | 22 (28.6) |
| PNI | | | | | | |
| No | 16 (61.5) | 18 (66.7) | 15 (60.0) | 0.872 | 33 (41.2) | 46 (59.0) |
| Yes | 10 (38.5) | 9 (33.3) | 10 (40.0) | | 47 (58.8) | 32 (41.0) |
| Subtype | | | | | | |
| Atypical | 19 (46.3) | 18 (42.9) | 11 (26.2) | 0.004 | 12 (11.7) | 20 (19.4) |
| Basal | 1 (2.4) | 5 (11.9) | 6 (14.3) | | 52 (50.5) | 36 (35.0) |
| Classical | 12 (29.3) | 17 (40.5) | 9 (21.4) | | 9 (8.7) | 21 (20.4) |
| Mesenchymal | 9 (22.0) | 2 (4.8) | 16 (38.1) | | 30 (29.1) | 26 (25.2) |

| | Oral Cavity | | HPV-positive Oropharynx | | | |
|---|---|---|---|---|---|---|
| | T3 | | T1 | T2 | T3 | |
| | (>0.411) | | (<−0.708) | (−0.708-0.013) | (>0.013) | |
| | 103 | | 16 | 15 | 16 | |
| | N (%) | p | N (%) | N (%) | N (%) | p |
| Gender | | | | | | |
| Female | 36 (35.0) | 0.826 | 1 (6.2) | 0 (0.0) | 4 (25.0) | 0.06 |
| Male | 67 (65.0) | | 15 (93.8) | 15 (100.0) | 12 (75.0) | |
| Race | | | | | | |
| Non-White | 21 (20.8) | 0.204 | 0 (0.0) | 1 (6.7) | 2 (12.5) | 0.35 |
| White | 80 (79.2) | | 16 (100.0) | 14 (93.3) | 14 (87.5) | |
| Smoking | | | | | | |
| Current | 27 (27.0) | 0.154 | 4 (25.0) | 0 (0.0) | 5 (31.2) | 0.23 |
| Former | 38 (38.0) | | 6 (37.5) | 7 (50.0) | 7 (43.8) | |
| Never | 35 (35.0) | | 6 (37.5) | 7 (50.0) | 4 (25.0) | |
| N Stage | | | | | | |
| N | 47 (48.0) | 0.132 | 3 (18.8) | 2 (13.3) | 4 (25.0) | 0.71 |
| N+ | 51 (52.0) | | 13 (81.2) | 13 (86.7) | 12 (75.0) | |
| T Stage | | | | | | |
| T1-T2 | 39 (39.4) | 0.616 | 11 (68.8) | 11 (73.3) | 9 (60.0) | 0.73 |
| T3-T4 | 60 (60.6) | | 5 (31.2) | 4 (26.7) | 6 (40.0) | |
| LVI | | | | | | |
| No | 53 (67.9) | 0.884 | 1 (16.7) | 1 (25.0) | 4 (66.7) | 0.17 |
| Yes | 25 (32.1) | | 5 (83.3) | 3 (75.0) | 2 (33.3) | |
| PNI | | | | | | |
| No | 30 (35.3) | 0.007 | 5 (83.3) | 5 (100.0) | 6 (100.0) | 0.38 |
| Yes | 55 (64.7) | | 1 (16.7) | 0 (0.0) | 0 (0.0) | |
| Subtype | | | | | | |
| Atypical | 3 (2.9) | <0.001 | 16 (100.0) | 15 (100.0) | 14 (87.5) | 0.13 |
| Basal | 40 (38.8) | | — | — | — | |
| Classical | 13 (12.6) | | 0 (0.0) | 0 (0.0) | 2 (12.5) | |
| Mesenchymal | 47 (45.6) | | — | — | — | |

TABLE 7
Hazard ratios for overall survival and disease-free survival in larynx cancer
for model 1) adjusting for LVI and PNI and model 2) excluding LVI and PNI

| | Overall Survival | | | | | | Disease free survival | | | | | |
|---|---|---|---|---|---|---|---|---|---|---|---|---|
| | Model 1 | | | Model 2 | | | Model 1 | | | Model 2 | | |
| | HR (95% CI) | | p-value | HR (95% CI) | | p-value | HR (95% CI) | | p-value | HR (95% CI) | | p-value |
| pEMT | | | | | | | | | | | | |
| T1 | 1.00 | | | 1 | | | 1.00 | | | 1 | | |
| T2 | 1.18 | (0.31, 4.48) | 0.803 | 1.4 | (0.62, 3.2) | 0.418 | 0.93 | (0.31, 2.77) | 0.90 | 1.09 | (0.53, 2.24) | 0.811 |
| T3 | 2.1 | (0.53, 8.23) | 0.289 | 1.7 | (0.72, 4) | 0.227 | 1.2 | (0.38, 3.82) | 0.76 | 1.25 | (0.6, 2.62) | 0.554 |
| Smoking | | | | | | | | | | | | |
| Current | 1.00 | | | 1 | | | 1.00 | | | 1 | | |
| Former | 0.47 | (0.16, 1.39) | 0.173 | 0.49 | (0.24, 1) | 0.050 | 0.86 | (0.33, 2.21) | 0.75 | 0.83 | (0.45, 1.53) | 0.552 |
| Never | 1.63 | (0.14, 19.75) | 0.699 | 4.32 | (1.28, 14.6) | 0.018 | 1.3 | (0.13, 13.48) | 0.82 | 3.21 | (0.98, 10.44) | 0.053 |
| T stage | | | | | | | | | | | | |
| T1-T2 | 1.00 | | | 1 | | | 1.00 | | | 1 | | |
| T3-T4 | 0.43 | (0.1, 1.8) | 0.247 | 0.85 | (0.36, 2.05) | 0.724 | 0.43 | (0.13, 1.45) | 0.18 | 1 | (0.45, 2.23) | 1.000 |
| N Stage | | | | | | | | | | | | |
| N0 | 1.00 | | | 1 | | | 1.00 | | | 1 | | |
| N+ | 1.41 | (0.4, 4.98) | 0.593 | 1.7 | (0.86, 3.35) | 0.125 | 1.18 | (0.42, 3.25) | 0.76 | 1.45 | (0.8, 2.63) | 0.217 |
| Race | | | | | | | | | | | | |
| Non-White | 1.00 | | | 1 | | | 1.00 | | | 1 | | |
| White | 0.54 | (0.15, 1.93) | 0.340 | 0.53 | (0.25, 1.13) | 0.102 | 0.5 | (0.17, 1.45) | 0.20 | 0.67 | (0.35, 1.29) | 0.230 |
| PNI | | | | | | | | | | | | |
| No | 1.00 | | | — | | | 1.00 | | | — | | |
| Yes | 2.42 | (0.72, 8.18) | 0.155 | — | | | 1.79 | (0.61, 5.2) | 0.29 | — | | |
| LVI | | | | | | | | | | | | |
| No | 1.00 | | | — | | | 1.00 | | | — | | |
| Yes | 2.04 | (0.58, 7.09) | 0.264 | — | | | 1.44 | (0.49, 4.22) | 0.51 | — | | |
| Age | 1.02 | (0.95, 1.09) | 0.590 | 1.01 | (0.97, 1.05) | 0.728 | 0.99 | (0.94, 1.05) | 0.79 | 0.99 | (0.96, 1.03) | 0.690 |

TABLE 8
Hazard ratios for overall survival and disease-free survival in oral cavity cancer for pEMT
by tertiles for model 1) adjusting for LVI and PNI and model 2) excluding LVI and PNI

| | Overall Survival | | | | Disease free survival | | | |
|---|---|---|---|---|---|---|---|---|
| | Model 1 | | Model 2 | | Model 1 | | Model 2 | |
| | HR (95% CI) | p-value | HR (95% CI) | p-value | HR (95% CI) | p-value | HR (95% CI) | p-value |
| pEMT | | | | | | | | |
| T1 | 1 | | 1 | | 1 | | 1 | |
| T2 | 0.69 (0.4, 1.2) | 0.193 | 0.58 (0.37, 0.93) | 0.022 | 0.75 (0.46, 1.24) | 0.267 | 0.71 (0.47, 1.07) | 0.101 |
| T3 | 1.28 (0.77, 2.11) | 0.336 | 1.19 (0.78, 1.8) | 0.414 | 1.13 (0.7, 1.8) | 0.622 | 1.13 (0.76, 1.68) | 0.544 |
| Smoking | | | | | | | | |
| Current | 1 | | 1 | | 1 | | 1 | |
| Former | 0.62 (0.36, 1.08) | 0.092 | 0.65 (0.42, 1.01) | 0.058 | 0.77 (0.46, 1.3) | 0.327 | 0.72 (0.47, 1.09) | 0.122 |
| Never | 0.5 (0.27, 0.93) | 0.028 | 0.61 (0.38, 0.99) | 0.046 | 0.7 (0.4, 1.23) | 0.213 | 0.8 (0.51, 1.24) | 0.321 |
| T stage | | | | | | | | |
| T1-T2 | 1 | | 1 | | 1 | | 1 | |
| T3-T4 | 1.16 (0.71, 1.9) | 0.549 | 1.27 (0.85, 1.91) | 0.245 | 1.23 (0.78, 1.92) | 0.370 | 1.32 (0.91, 1.91) | 0.148 |
| N Stage | | | | | | | | |
| N0 | 1 | | 1 | | 1 | | 1 | |
| N+ | 1.26 (0.77, 2.06) | 0.361 | 1.25 (0.85, 1.84) | 0.259 | 1.18 (0.76, 1.86) | 0.462 | 1.18 (0.83, 1.68) | 0.359 |
| Race | | | | | | | | |
| Non-White | 1 | | 1 | | 1 | | 1 | |
| White | 0.85 (0.47, 1.54) | 0.586 | 0.79 (0.47, 1.33) | 0.377 | 0.79 (0.47, 1.35) | 0.394 | 0.79 (0.49, 1.26) | 0.326 |
| PNI | | | | | | | | |
| No | 1 | | — | | 1 | | — | |
| Yes | 1.8 (1.13, 2.87) | 0.014 | — | | 1.56 (1.03, 2.38) | 0.037 | — | |
| LVI | | | | | | | | |
| No | 1 | | — | | 1 | | — | |
| Yes | 1.65 (1.04, 2.61) | 0.033 | — | | 1.69 (1.11, 2.58) | 0.015 | — | |
| Age | 1.04 (1.02, 1.06) | 0.000 | 1.03 (1.01, 1.04) | 0.003 | 1.03 (1.02, 1.05) | 0.000 | 1.02 (1.01, 1.04) | 0.001 |

TABLE 9
Hazard ratios for overall survival and disease-free survival in larynx cancer for pEMT by high and low as
determined by cutpoint analysis for model 1) adjusting for LVI and PNI and model 2) excluding LVI and PNI

| | Overall Survival | | | | Disease free survival | | | |
|---|---|---|---|---|---|---|---|---|
| | Model 1 | | Model 2 | | Model 1 | | Model 2 | |
| | HR (95% CI) | p-value | HR (95% CI) | p-value | HR (95% CI) | p-value | HR (95% CI) | p-value |
| pEMT | | | | | | | | |
| Low | 1 | | 1 | | 1 | | 1 | |
| High (>0.6169) | 4.59 (0.94, 22.4) | 0.060 | 3.96 (1.75, 8.95) | 0.001 | 1.77 (0.4, 7.9) | 0.456 | 2.80 (1.36, 5.75) | 0.005 |
| Smoking | | | | | | | | |
| Current | 1 | | 1 | | 1 | | 1 | |
| Former | 0.41 (0.14, 1.24) | 0.114 | 0.52 (0.26, 1.04) | 0.064 | 0.84 (0.32, 2.15) | 0.708 | 0.83 (0.46, 1.51) | 0.539 |
| Never | 1.12 (0.08, 15.4) | 0.932 | 2.82 (0.85, 9.39) | 0.091 | 1.13 (0.10, 12.5) | 0.923 | 2.13 (0.66, 6.83) | 0.204 |
| T stage | | | | | | | | |
| T1-T2 | 1 | | 1 | | 1 | | 1 | |
| T3-T4 | 0.65 (0.13, 3.2) | 0.595 | 1.09 (0.44, 2.7) | 0.855 | 0.5 (0.14, 1.85) | 0.3 | 1.20 (0.52, 2.75) | 0.668 |
| N Stage | | | | | | | | |
| N0 | 1 | | 1 | | 1 | | 1 | |
| N+ | 1.51 (0.44, 5.13) | 0.513 | 1.65 (0.84, 3.25) | 0.149 | 1.26 (0.46, 3.44) | 0.657 | 1.48 (0.82, 2.69) | 0.194 |
| Race | | | | | | | | |
| Non-White | 1 | | 1 | | 1 | | 1 | |
| White | 0.39 (0.11, 1.4) | 0.150 | 0.54 (0.26, 1.13) | 0.104 | 0.47 (0.16, 1.36) | 0.164 | 0.67 (0.35, 1.28) | 0.222 |
| PNI | | | | | | | | |
| No | 1 | | — | | 1 | | — | |
| Yes | 1.97 (0.57, 6.81) | 0.282 | — | | 1.62 (0.53, 4.92) | 0.396 | — | |
| LVI | | | | | | | | |
| No | 1 | | — | | 1 | | — | |
| Yes | 2.21 (0.63, 7.83) | 0.218 | — | | 1.44 (0.50, 4.18) | 0.5 | — | |
| Age | 1.03 (0.97, 1.11) | 0.348 | 1.02 (0.98, 1.07) | 0.337 | 1.00 (0.94, 1.06) | 0.931 | 1.00 (0.97, 1.04) | 0.890 |

TABLE 10
Hazard ratios for overall survival and disease-free survival in oral cavity cancer for pEMT by high and low
as determined by cutpoint analysis for model 1) adjusting for LVI and PNI and model 2) excluding LVI and PNI

| | Overall Survival | | | | Disease free survival | | | |
|---|---|---|---|---|---|---|---|---|
| | Model 1 | | Model 2 | | Model 1 | | Model 2 | |
| | HR (95% CI) | p-value | HR (95% CI) | p-value | HR (95% CI) | p-value | HR (95% CI) | p-value |
| pEMT | | | | | | | | |
| Low | 1 | | 1 | | 1 | | 1 | |
| High (>−0.3877) | 1.45 (0.89, 2.37) | 0.134 | 1.62 (1.06, 2.46) | 0.024 | 1.29 (0.83, 2.01) | 0.262 | 1.4 (0.96, 2.03) | 0.081 |
| Smoking | | | | | | | | |
| Current | 1 | | 1 | | 1 | | 1 | |
| Former | 0.65 (0.38, 1.12) | 0.122 | 0.66 (0.43, 1.03) | 0.065 | 0.78 (0.47, 1.3) | 0.338 | 0.72 (0.48, 1.1) | 0.127 |
| Never | 0.54 (0.29, 0.99) | 0.044 | 0.65 (0.4, 1.05) | 0.075 | 0.73 (0.42, 1.26) | 0.254 | 0.83 (0.54, 1.29) | 0.408 |
| T stage | | | | | | | | |
| T1-T2 | 1 | | 1 | | 1 | | 1 | |
| T3-T4 | 1.17 (0.72, 1.91) | 0.535 | 1.24 (0.83, 1.87) | 0.291 | 1.24 (0.79, 1.94) | 0.352 | 1.3 (0.9, 1.89) | 0.163 |
| N Stage | | | | | | | | |
| N0 | 1 | | 1 | | 1 | | 1 | |
| N+ | 1.28 (0.78, 2.09) | 0.333 | 1.27 (0.86, 1.86) | 0.232 | 1.19 (0.76, 1.87) | 0.451 | 1.19 (0.83, 1.69) | 0.345 |
| Race | | | | | | | | |
| Non-White | 1 | | 1 | | 1 | | 1 | |
| White | 0.86 (0.48, 1.55) | 0.611 | 0.79 (0.47, 1.32) | 0.364 | 0.8 (0.47, 1.36) | 0.410 | 0.79 (0.49, 1.25) | 0.309 |
| PNI | | | | | | | | |
| No | 1 | | — | | | | — | |
| Yes | 1.85 (1.16, 2.95) | 0.009 | — | | 1.6 (1.05, 2.43) | 0.028 | — | |
| LVI | | | | | | | | |
| No | 1 | | — | | 1 | | — | |
| Yes | 1.64 (1.04, 2.58) | 0.035 | — | | 1.69 (1.11, 2.58) | 0.015 | — | |
| Age | 1.04 (1.02, 1.06) | 0.001 | 1.02 (1.01, 1.04) | 0.005 | 1.03 (1.01, 1.05) | 0.001 | 1.02 (1.01, 1.04) | 0.002 |

TABLE 11
Adjusted hazard ratio for overall survival and disease-free survival for larynx and oral cavity cancer in the imputed dataset.

| | Larynx Cancer | | | | Oral Cavity Cancer | | | | |
|---|---|---|---|---|---|---|---|---|---|
| | Overall Survival | | Disease-free survival | | | Overall Survival | | Disease-free survival | |
| | HR (95% CI) | p | HR (95% CI) | p | | HR (95% CI) | p | HR (95% CI) | p |
| pEMT | | | | | pEMT | | | | |
| Low | 1 | | 1 | | Low | 1 | | 1 | |
| High (>0.62) | 4.26 (1.90, 9.55) | <0.001 | 3.21 (1.58, 6.49) | 0.001 | High (>−0.39) | 1.27 (0.87, 1.86) | 0.222 | 1.45 (0.95, 2.2) | 0.086 |
| Smoking | | | | | Smoking | | | | |
| Current | 1 | | 1 | | Current | 1 | | 1 | |
| Former/Never | 0.88 (0.35, 2.23) | 0.789 | 1.09 (0.48, 2.45) | 0.839 | Former | 1.2 (0.81, 1.77) | 0.364 | 1.02 (0.68, 1.55) | 0.909 |
| T stage | | | | | T stage | | | | |
| T1-T2 | 1 | | 1 | | T1-T2 | 1 | | 1 | |
| T3-T4 | 0.59 (0.3, 1.15) | 0.121 | 0.91 (0.5, 1.65) | 0.757 | T3-T4 | 0.79 (0.54, 1.17) | 0.244 | 0.65 (0.43, 0.99) | 0.044 |
| N Stage | | | | | N Stage | | | | |
| N0 | 1 | | 1 | | N0 | 1 | | 1 | |
| N+ | 1.26 (0.57, 2.78) | 0.568 | 1.18 (0.6, 2.35) | 0.631 | N+ | 1.08 (0.73, 1.6) | 0.699 | 1.13 (0.74, 1.71) | 0.578 |
| Race | | | | | Race | | | | |
| Non-White | 1 | | 1 | | Non-White | 1 | | 1 | |
| White | 0.57 (0.24, 1.37) | 0.208 | 0.75 (0.35, 1.58) | 0.442 | White | 0.53 (0.29, 0.97) | 0.041 | 0.56 (0.29, 1.07) | 0.078 |
| PNI | | | | | PNI | | | | |
| No | 1 | | 1 | | No | 1 | | 1 | |
| Yes | 1.02 (0.98, 1.06) | 0.306 | 1.39 (0.58, 3.33) | 0.462 | Yes | 1.03 (1.01, 1.04) | 0.001 | 1.41 (0.92, 2.17) | 0.115 |
| LVI | | | | | LVI | | | | |
| No | 1 | | 1 | | No | 1 | | 1 | |
| Yes | 1.72 (0.6, 4.92) | 0.315 | 1.02 (0.43, 2.41) | 0.972 | Yes | 1.38 (0.92, 2.07) | 0.123 | 1.46 (0.91, 2.33) | 0.115 |
| Age | 1.2 (0.46, 3.13) | 0.715 | 1.01 (0.97, 1.04) | 0.733 | Age | 1.43 (0.92, 2.20) | 0.109 | 1.02 (1.01, 1.04) | 0.007 |

TABLE 12
Interaction between race and pEMT for overall
survival and disease-free survival.

| | Overall survival | | Disease-free survival | |
|---|---|---|---|---|
| | HR (95% CI) | p | HR (95% CI) | p |
| pEMT:Race | | | | |
| Low:White | 1 | | 1 | |
| Low:Black | 0.96 (0.38, 2.43) | 0.922 | 1.06 (0.50, 2.23) | 0.818 |
| High:White | 1.68 (1.12, 2.52) | 0.013 | 1.47 (1.02, 2.13) | 0.077 |
| High:Black | 4.14 (2.17, 7.91) | 0.000 | 3.78 (2.09, 6.82) | <0.001 |
| Smoking | | | | |
| Current | 1 | | 1 | |
| Former/Never | 0.68 (0.48, 0.97) | 0.031 | 0.88 (0.64, 1.2) | 0.206 |
| T stage | | | | |
| T1-T2 | 1 | | 1 | |
| T3-T4 | 0.97 (0.67, 1.41) | 0.874 | 1.14 (0.81, 1.61) | 0.327 |
| N Stage | | | | |
| N0 | 1 | | 1 | |
| N+ | 1.14 (0.79, 1.64) | 0.495 | 1.11 (0.79, 1.54) | 0.557 |
| PNI | | | | |
| No | 1 | | 1 | |
| Yes | 1.48 (1.01, 2.16) | 0.045 | 1.34 (0.95, 1.89) | 0.066 |
| LVI | | | | |
| No | 1 | | 1 | |
| Yes | 1.39 (0.92, 2.09) | 0.117 | 1.30 (0.89, 1.89) | 0.177 |
| Age | 1.02 (1.01, 1.04) | 0.009 | 1.02 (1.01, 1.04) | 0.006 |
| Site | | | | |
| Larynx | 1 | | 1 | |
| Oral Cavity | 1.09 (0.71, 1.69) | 0.688 | 1.06 (0.71, 1.57) | 0.846 |

TABLE 13
Hazard ratios for overall survival and disease-free survival in larynx cancer for pEMT by high and low as determined by
cutpoint analysis for model 1) adjusting for LVI and PNI and model 2) excluding LVI and PNI using complete case analysis.

| | Overall Survival | | | | Disease free survival | | | |
|---|---|---|---|---|---|---|---|---|
| | Model 1 | | Model 2 | | Model 1 | | Model 2 | |
| | HR (95% CI) | p-value | HR (95% CI) | p-value | HR (95% CI) | p-value | HR (95% CI) | p-value |
| pEMT | | | | | | | | |
| Low | 1 | | 1 | | 1 | | 1 | |
| High (>0.6169) | 5.03 (1.1, 23.04) | 0.037 | 4.89 (2.27, 10.55) | <0.001 | 1.84 (0.44, 7.75) | 0.403 | 3.20 (1.62, 6.33) | 0.001 |
| Smoking | | | | | | | | |
| Current | 1 | | 1 | | 1 | | 1 | |
| Former | 0.46 (0.16, 1.3) | 0.145 | 0.65 (0.35, 1.23) | 0.187 | 0.86 (0.35, 2.13) | 0.747 | 0.92 (0.52, 1.63) | 0.776 |
| T stage | | | | | | | | |
| T1-T2 | 1 | | 1 | | 1 | | 1 | |
| T3-T4 | 0.56 (0.13, 2.3) | 0.418 | 0.96 (0.4, 2.3) | 0.934 | 0.49 (0.14, 1.71) | 0.261 | 1.12 (0.5, 2.52) | 0.781 |
| N Stage | | | | | | | | |
| N0 | 1 | | 1 | | 1 | | 1 | |
| N+ | 1.46 (0.44, 4.84) | 0.535 | 1.66 (0.85, 3.24) | 0.136 | 1.26 (0.46, 3.43) | 0.656 | 1.51 (0.84, 2.73) | 0.169 |
| Race | | | | | | | | |
| Non-White | 1 | | 1 | | 1 | | 1 | |
| White | 0.38 (0.10, 1.38) | 0.141 | 0.63 (0.30, 1.29) | 0.206 | 0.47 (0.16, 1.36) | 0.163 | 0.72 (0.38, 1.36) | 0.311 |
| PNI | | | | | | | | |
| No | 1 | | — | | 1 | | — | |
| Yes | 1.65 (0.54, 5.03) | 0.376 | — | | 1.54 (0.55, 4.32) | 0.410 | — | |
| LVI | | | | | | | | |
| No | 1 | | — | | 1 | | — | |
| Yes | 2.52 (0.75, 8.48) | 0.135 | — | | 1.48 (0.52, 4.19) | 0.457 | — | |
| Age | 1.03 (0.97, 1.11) | 0.339 | 1.02 (0.98, 1.07) | 0.306 | 1.00 (0.94, 1.06) | 0.928 | 1.00 (0.97, 1.04) | 0.866 |

TABLE 14
Hazard ratios for overall survival and disease-free survival in oral cavity cancer for pEMT by high and low as determined by
cutpoint analysis for model 1) adjusting for LVI and PNI and model 2) excluding LVI and PNI using complete case analysis.

| | Overall Survival | | | | Disease free survival | | | |
|---|---|---|---|---|---|---|---|---|
| | Model 1 | | Model 2 | | Model 1 | | Model 2 | |
| | HR (95% CI) | p-value | HR (95% CI) | p-value | HR (95% CI) | p-value | HR (95% CI) | p-value |
| pEMT | | | | | | | | |
| Low | 1 | | 1 | | 1 | | 1 | |
| High (>−0.3877) | 1.44 (0.88, 2.34) | 0.147 | 1.62 (1.07, 2.45) | 0.024 | 1.28 (0.82, 2.00) | 0.269 | 1.42 (0.98, 2.06) | 0.068 |
| Smoking | | | | | | | | |
| Current | 1 | | 1 | | 1 | | 1 | |
| Former/Never | 0.60 (0.37, 1.00) | 0.049 | 0.66 (0.44, 0.98) | 0.037 | 0.76 (0.47, 1.21) | 0.246 | 0.77 (0.53, 1.12) | 0.168 |
| T stage | | | | | | | | |
| T1-T2 | 1 | | 1 | | 1 | | 1 | |
| T3-T4 | 1.18 (0.73, 1.93) | 0.502 | 1.25 (0.83, 1.87) | 0.289 | 1.24 (0.8, 1.94) | 0.340 | 1.3 (0.89, 1.88) | 0.173 |
| N Stage | | | | | | | | |
| N0 | 1 | | 1 | | 1 | | 1 | |
| N+ | 1.26 (0.77, 2.06) | 0.352 | 1.27 (0.86, 1.86) | 0.231 | 1.19 (0.76, 1.86) | 0.458 | 1.18 (0.83, 1.69) | 0.358 |
| Race | | | | | | | | |
| Non-White | 1 | | 1 | | 1 | | 1 | |
| White | 0.84 (0.46, 1.5) | 0.55 | 0.79 (0.47, 1.32) | 0.364 | 0.79 (0.47, 1.34) | 0.387 | 0.78 (0.49, 1.25) | 0.301 |
| PNI | | | | | | | | |
| No | 1 | | — | | 1 | | — | |
| Yes | 1.86 (1.17, 2.96) | 0.009 | — | | 1.6 (1.05, 2.43) | 0.028 | — | |
| LVI | | | | | | | | |
| No | 1 | | — | | 1 | | — | |
| Yes | 1.66 (1.05, 2.62) | 0.03 | — | | 1.7 (1.12, 2.59) | 0.014 | — | |
| Age | 1.04 (1.02, 1.06) | 0 | 1.02 (1.01, 1.04) | 0.005 | 1.03 (1.01, 1.05) | 0 | 1.02 (1.01, 1.04) | 0.003 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of treating an epithelial cancer in a subject in need thereof comprising:
determining the subject has a high or low risk of death, recurrence, or metastasis for an epithelial cancer by:
determining an average expression level of one or more partial EMT-like (p-EMT) signature genes or polypeptides in malignant cells of the epithelial cancer from the subject, wherein the one or more p-EMT signature genes or polypeptides are selected from the group consisting of SERPINE1, TGFB1, MMP10, LAMC2, P4HA2, PDPN, ITGA5, LAMA3, CH13, TNC, MMP2, EMP3, INHBA, LAMB3, SNAIL2, and VIM; and
comparing the average expression level of the p-EMT signature genes or polypeptides from the subject to a control average expression level of the p-EMT signature genes or polypeptides in malignant cells of an epithelial cancer obtained from a plurality of subjects having the epithelial cancer and belonging to the same demographic group as the subject, wherein the subject is determined as having high risk if the average expression level of one or more p-EMT signature genes or polypeptides from the subject is higher than the control average expression level, and the subject is determined as having low risk if the average expression level of the p-EMT signature genes or polypeptides from the subject is lower than the control average expression level; and
treating the low risk subject with an immunotherapy, a neoadjuvant therapy, a chemoradiation, or any combination thereof; or
treating the high risk subject with a lymph node dissection, an adjuvant chemotherapy, an adjuvant radiation or a post-operative radiation treatment (PORT), a chemoradiation, an agent that inhibits TGF beta signaling; one or more agents targeting malignant cells expressing a p-EMT signature, or any combination thereof, optionally, in combination with an immunotherapy, a neoadjuvant therapy a chemoradiation, or any combination thereof.

2. The method of claim 1, wherein the demographic group is selected from the group consisting of African American, Caucasian, non-Caucasian, non-smoker, current smoker, former smoker, male, and female.

3. The method of claim 1, wherein the control average expression level is the median average expression level of the one or more p-EMT signature genes or polypeptides for malignant cells of an epithelial cancer obtained from the plurality of subjects within fer the demographic group; or wherein the control average expression level is an intermediate average expression level of the one or more p-EMT signature genes or polypeptides within the range of average expression level for malignant cells of an epithelial cancer obtained from the plurality of subjects within the demographic group.

4. The method of claim 1, wherein the average expression level is determined by RNA sequencing (RNA-seq), or by immunohistochemistry (IHC).

5.-8. (canceled)

9. The method of claim 1, wherein determining the average expression level further comprises determining the percentage of cells having an average expression level higher than the control average expression level, wherein the subject is determined as having high risk if the percentage of cells having a higher average expression level is greater than the control percentage and the subject is determined as having low risk if the percentage of cells having a higher average expression level is lower than the control percentage.

10. The method of claim 1, further comprising determining a p-EMT score for the subject,
wherein the p-EMT score is a difference between the average expression level of the one or more p-EMT signature genes or polypeptides and an average expression level of a control gene set for the subject,
wherein the control gene set comprises genes having a similar distribution of expression levels as the control average expression level for each p-EMT signature gene or polypeptide,
wherein a score greater than zero indicates high p-EMT score, and a score less than zero indicates low p-EMT score, and
wherein the subject is determined as having high risk if a high p-EMT score is determined and the subject is determined as having low risk if a low p-EMT score is determined.

11. The method of claim 10, wherein the control gene set has at least 20-100 genes for each p-EMT gene.

12. The method of claim 10, wherein:
a high p-EMT score is greater than 0.5, and a low p-EMT score is less than −0.5 for any demographic selected from the group consisting of Caucasian, non-smoker, and female;
a high p-EMT score is greater than 0.4, and a low p-EMT score is less than −0.4 for non-Caucasians;
a high p-EMT score is greater than 0.3, and a low p-EMT score is less than −0.3 for males;
a high p-EMT score is greater than 0.2, and a low p-EMT score is less than −0.2 for African Americans; and
a high p-EMT score is greater than 0.1, and a low p-EMT score is less than −0.1 for African American males.

13.-16. (canceled)

17. The method of claim 1, wherein the subject has a clinically NO (cNO) neck.

18. (canceled)

19. The method of claim 1, wherein the subject is older than 35, 40, 45, 50, 55 or 60 years.

20. The method of claim 1, wherein the subject has been diagnosed as having human papillomavirus (HPV).

21.-40. (canceled)

41. The method of claim 1, wherein the subject determined as having high risk has decreased survival, increased risk for occult nodal metastasis, or increased risk for perineural invasion (PNI) as compared to the subject determined as having low risk.

42. The method of claim 41, wherein the subject determined as having high risk is at least twice as likely to die in a 15 year period as compared to the subject determined as having low risk.

43.-44. (canceled)

45. The method of claim 1, wherein chemoradiation comprises cisplatin.

46. The method of claim 1, wherein the immunotherapy comprises checkpoint blockade therapy.

47. The method of claim 1, further comprising monitoring the subject, wherein the subject is undergoing treatment for an epithelial cancer, comprising determining whether the p-EMT signature or p-EMT score increases or decreases in the subject during the treatment.

48. The method of claim 47, wherein the treatment comprises an agent that inhibits TGF beta signaling.

49. A method for identifying an agent capable of modulating or shifting a p-EMT signature comprising:
a. applying a candidate agent to a cell or population of cells having a p-EMT signature comprising one or more genes or polypeptides selected from the group consisting of SERPINE1, TGFBI, MMP10, LAMC2, P4HA2, PDPN, ITGA5, LAMA3, CDH13, TNC, MMP2, EMP3, INHBA, LAMB3, SNAIL2 and VIM; and
b. detecting modulation of the p-EMT signature for the cell or cell population by the candidate agent, wherein the p-EMT signature is detected by a method comprising:
detecting a change in an average expression of one or more partial EMT-like (p-EMT) signature genes or polypeptides in the cell or cell population treated with a candidate agent; and
identifying the candidate agent as an agent capable of modulating or shifting a p-EMT signature, if a change in the p-EMT signature is detected.

50. The method of claim 1, wherein the epithelial cancer is selected from the group consisting of head and neck cancer (HNSCC), lung, breast, prostate, colon, cutaneous squamous cell carcinoma and esophageal carcinoma.

51. The method of claim 50, wherein the epithelial cancer is head and neck cancer (HNSCC).
